(19) United States
(12) Patent Application Publication
(10) Pub. No.: US 2025/0085569 A1
(43) Pub. Date: Mar. 13, 2025

(54) ASPHERIC PHASE-RING STRUCTURED LENS DESIGNS, MANUFACTURE, AND USES THEREOF

(71) Applicant: AST Products, Inc., Billerica, MA (US)

(72) Inventors: Yi-Feng CHIU, Hsinchu (TW); Chuan-Hui YANG, Taichung (TW); Wen-Chu TSENG, Westford, MA (US)

(73) Assignee: AST Products, Inc., Billerica, MA (US)

(21) Appl. No.: 18/728,833

(22) PCT Filed: Jan. 13, 2023
(86) PCT No.: PCT/US2023/060681

§ 371 Date: Jul. 12, 2024

Related U.S. Application Data
(60) Provisional application No. 63/299,635, filed on Jan. 14, 2022.

(51) Int. Cl. G02C 7/02; G02C 7/06

(52) U.S. Cl. CPC G02C 7/022; G02C 7/06

(57) ABSTRACT

Described herein are ophthalmic lenses with double-sided aspherical optics containing phase¬ring structures, such as extended depth of focus (EDOF) ophthalmic lenses and multifocal lenses. Refraction of light passing through one or more regions of the phase-ring structures described herein can cause constructive interference, thereby individually or as a set producing an extended depth of focus and improved distance focus, intermediate focus, and near focus. The ophthalmic lenses described herein may provide improved vision acuity and enhanced contrast and reduce or remove visual effects such as dysphotopsia (e.g., halos and glare).

The application claims benefit of priority under 35 U.S.C. § 119(e) of U.S. Ser. No. 63/299,635, filed Jan. 14, 2022, the content of which is incorporated by reference in its entirety.

### FIELD

The present disclosure relates generally to ophthalmic lenses, and more specifically to novel double-sided aspheric phase-ring structured lenses, designs, manufacture, and uses thereof.

### BACKGROUND

Ophthalmology is the field of medicine directed to the anatomy, physiology, and diseases of the human eye. The anatomy of the human eye is rather complex. The main structures of the eye include the cornea, an aspherical clear tissue at the outer front of the eye; the iris, which is the colored part of the eye; the pupil, an adaptable aperture in the iris that regulates the amount of light received in the eye; the crystalline lens, a small clear disk inside the eye that focuses light rays onto the retina; and the retina, a layer that forms the rear or backside of the eye and transforms sensed light into electrical impulses that travel through the optic nerve to the brain. The posterior chamber (i.e., the space between the retina and the lens) is filled with aqueous humour, and the anterior chamber (i.e., the space between the lens and the cornea) is filled with vitreous humour, or a clear, jelly-like substance.

The natural crystalline lens has a flexible, transparent, biconvex structure, and together with the cornea, operates to refract light to be focused on the retina. The lens is flatter on its anterior side than on its posterior side and its curvature is controlled by the ciliary muscles to which the lens connects by suspensory ligaments, called zonules. By changing the curvature of the lens, the focal distance of the eye is changed so as to focus on objects at various distances. To view an object at a short distance from the eye, the ciliary muscles contract, and the lens thickens, resulting in a rounder shape and thus high refractive power. Changing focus to an object at a greater distance requires the relaxation of the lens and thus increasing the focal distance. This process of changing curvature and adapting the focal distance of the eye to form a sharp image of an object at the retina is called accommodation.

In humans, the refractive power of the crystalline lens in its natural environment is approximately 18-20 diopters, roughly one-third of the total optical power of the eye. The cornea provides the remaining 40 diopters of the total optical power of the eye.

With the ageing of the eye, the opaqueness of the lens increases, called a cataract. Some diseases like diabetes, trauma, some medications, and excessive UV light exposure may also cause a cataract. A cataract is painless and results in a cloudy, blurry vision. Treatments for cataracts include surgery, by which the cloudy lens is removed and replaced with an artificial one, generally called an intraocular lens (IOL or IOLs).

Another age-related effect is called presbyopia, which is manifested by difficulty in reading small print or seeing nearby pictures clearly. Presbyopia generally is believed to be caused by a thickening and loss of flexibility of the natural lens inside the eye. Age-related changes also take place in the ciliary muscles surrounding the lens. With less elasticity it becomes harder to focus on objects close to the eye.

A variety of intraocular lenses are also employed for correcting other visual disorders, such as myopia, or nearsightedness when the eye is unable to see distant objects caused by, for example, the cornea having too much curvature. The effect of myopia is that distant light rays focus on a point in front of the retina, rather than directly on its surface. Hyperopia, or farsightedness caused by an abnormally flat cornea, causes light rays entering the eye to focus behind the retina, therefore not allowing the eye to see objects that are close. Astigmatism is another common cause of visual difficulty in which images are blurred due to an irregularly shaped cornea.

In the majority of cases, intraocular lenses (IOLs) are implanted in a patient's eye during cataract surgery to replace the natural crystalline lens and compensate for the loss of optical power of the removed lens. Modern IOL optics are designed to have a multifocal optic for providing short, intermediary and distance vision of objects, also called multifocal IOLs, or more specifically, trifocal lenses. Presbyopia is traditionally corrected by eyeglasses or contact lenses, and patients may also opt for multifocal optics. In some cases, an IOL can include diffractive structures to have not only a far-focus power but also a near-focus power, thereby providing a degree of pseudo-accommodation. However, a variety of aberrations, such as spherical and astigmatic aberrations, can adversely affect the optical performance of such lenses. For example, spherical aberrations can degrade vision contrast, especially for large pupil sizes.

Although multifocal ophthalmic lenses often lead to improved quality of vision for many patients, additional improvements would be beneficial. For example, some pseudophakic patients experience undesirable visual effects (dysphotopsia), such as glare or halos, especially during use in dark environments (e.g., nighttime). Halos may arise when light from the unused focal image creates an out-of-focus image that is superimposed on the used focal image. For example, if light from a distant point source is imaged onto the retina by the distant focus of a bifocal IOL, the near focus of the IOL will simultaneously superimpose a defocused image on top of the image formed by the distant focus. This defocused image may manifest itself in the form of a ring of light surrounding the in-focus image and is referred to as a halo. Another area in need of improvement revolves around the typical bifocality of multifocal lenses. More particularly, since multifocal ophthalmic lenses typically provide for near and far vision, intermediate vision may be compromised.

Accordingly, what is needed is one or more ophthalmic lenses with an extended depth of focus (EDOF) that may simultaneously provide a near focus, intermediate focus, and distance focus, while also addressing the aforementioned adverse effects, such as dysphotopsia (e.g., halo, glare, etc.), thereby providing enhanced contrast and improved visual acuity.

### SUMMARY

The present disclosure is related to double-sided aspheric ophthalmic lenses with phase-ring structures, which can provide an extended depth of focus, thereby enhancing contrast and improving vision acuity while reducing undesirable visual effects such as glare and halos. The phase-ring structures of the ophthalmic lenses described herein may be designed to provide depth of focus via a non-diffracting beam principle with constructive phasing, thereby maximizing the focal energy in the lens.

In some embodiments, an ophthalmic lens comprising a lens body is provided, the lens body comprising: a first aspheric surface; and a second aspheric surface comprising a base curvature and a phase-ring structure, wherein the phase-ring structure comprises an inner region and an outer region, and the inner region comprises a first phase-ring surface having a first curvature; the outer region comprises a second phase-ring surface having a second curvature; and an outer edge of the inner region is adjacent to an inner edge of the outer region, wherein refraction of light passing through the inner region and the outer region causes constructive interference that produces a distance focus and an extended focus.

In some embodiments, a method of treating an ophthalmic disease or disorder in a subject is provided, the method comprising implanting into an eye of the subject an ophthalmic lens comprising a lens body, the lens body comprising: a first aspheric surface; and a second aspheric surface comprising a base curvature and a phase-ring structure, wherein the phase-ring structure comprises an inner region and an outer region, and the inner region comprises a first phase-ring surface having a first curvature; the outer region comprises a second phase-ring surface having a second curvature; and an outer edge of the inner region is adjacent to an inner edge of the outer region, wherein refraction of light passing through the inner region and the outer region causes constructive interference that produces a distance focus and an extended focus.

In some embodiments, a method of manufacturing an ophthalmic lens is provided, the method comprising: manufacturing a first aspheric surface; manufacturing a second aspheric surface comprising a base curvature; and generating a phase-ring structure on the second aspheric surface, wherein the phase-ring structure comprises an inner region and an outer region, and the inner region comprises a first phase-ring surface having a first curvature; the outer region comprises a second phase-ring surface having a second curvature; and an outer edge of the inner region is adjacent to an inner edge of the outer region, wherein refraction of light passing through the inner region and the outer region causes constructive interference that produces a distance focus and an extended focus.

In some embodiments, an ophthalmic lens comprising a lens body is provided, the lens body comprising: a first aspheric surface; and a second aspheric surface comprising a base curvature and a phase-ring structure, wherein the phase-ring structure comprises an inner region, an intermediate region, and an outer region, and the inner region comprises a first phase-ring surface having a first curvature; the intermediate region comprises a second phase-ring surface having a second curvature; the outer region comprises a third phase-ring surface having a third curvature; an outer edge of the inner region is adjacent to an inner edge of the intermediate region; and an outer edge of the intermediate region is adjacent to an inner edge of the outer region, wherein refraction of light passing through the inner region, the intermediate region, and the outer region causes constructive interference that produces a near focus and a distance focus.

In some embodiments, a method of treating an ophthalmic disease or disorder in a subject, the method comprising implanting into an eye of the subject an ophthalmic lens comprising a lens body, the lens body comprising: a first aspheric surface; and a second aspheric surface comprising a base curvature and a phase-ring structure, wherein the phase-ring structure comprises an inner region, an intermediate region, and an outer region, and the inner region comprises a first phase-ring surface having a first curvature; the intermediate region comprises a second phase-ring surface having a second curvature; the outer region comprises a third phase-ring surface having a third curvature; an outer edge of the inner region is adjacent to an inner edge of the intermediate region; and an outer edge of the intermediate region is adjacent to an inner edge of the outer region, wherein refraction of light passing through the inner region, the intermediate region, and the outer region causes constructive interference that produces a near focus and a distance focus.

In some embodiments, a method of manufacturing an ophthalmic lens is provided, the method comprising: manufacturing a first aspheric surface; manufacturing a second aspheric surface comprising a base curvature; and generating a phase-ring structure on the second aspheric surface, wherein the phase-ring structure comprises an inner region, an intermediate region, and an outer region, and the inner region comprises a first phase-ring surface having a first curvature; the intermediate region comprises a second phase-ring surface having a second curvature; the outer region comprises a third phase-ring surface having a third curvature; an outer edge of the inner region is adjacent to an inner edge of the intermediate region; and an outer edge of the intermediate region is adjacent to an inner edge of the outer region, wherein refraction of light passing through the inner region, the intermediate region, and the outer region causes constructive interference that produces a near focus and a distance focus.

In some embodiments, a set of ophthalmic lenses is provided, comprising: a first ophthalmic lens comprising a first lens body, the first lens body comprising: a first aspheric surface; and a second aspheric surface comprising a first base curvature and a first optical phase-ring structure, wherein the first optical phase-ring structure comprises a first inner region and a first outer region, and the first inner region comprises a first phase-ring surface; and the outer region comprises a second phase-ring surface; a second ophthalmic lens comprising a second lens body, the second lens body comprising: a third aspheric surface; and a fourth aspheric surface comprising a second base curvature and a second optical phase-ring structure, wherein the second optical phase-ring structure comprises a second inner region, an intermediate region, and a second outer region, and the second inner region comprises a third phase-ring surface; the intermediate region comprises a fourth phase-ring surface; and the second outer region comprises a fifth phase-ring surface; wherein refraction of light passing through the first inner region and the outer region of the first ophthalmic lens causes first constructive interference and refraction of light passing through the second inner region, the intermediate region, and the second outer region causes second constructive interference, thereby collectively producing a near focus, distance focus, and an extended focus.

### BRIEF DESCRIPTION OF THE DRAWINGS

The present application can be best understood by reference to the following description taken in conjunction with the accompanying figures included in the specification.

FIG. 1 illustrates the height profile of an aspherical base structure of a double-sided aspheric ophthalmic lens with a phase-ring structure, according to some embodiments of the present disclosure;

FIG. 2A illustrates a phase profile of the phase-ring structure of an extended depth of focus (EDOF) ophthalmic lens, according to a first embodiment of the present disclosure;

FIG. 2B illustrates the phase profile of the phase-ring structure of the EDOF ophthalmic lens, according to a second embodiment of the present disclosure;

FIG. 2C illustrates the phase profile of the phase-ring structure of the EDOF ophthalmic lens, according to a third embodiment of the present disclosure;

FIG. 3 illustrates the total height profile of an aspherical surface with a phase-ring structure of the EDOF ophthalmic lens, according to some embodiments of the present disclosure;

FIG. 4 illustrates an optical performance (modulation transfer function, MTF) of the EDOF ophthalmic lens, according to a first embodiment of the present disclosure;

FIG. 5 illustrates the optical performance (MTF) of the EDOF ophthalmic lens, according to a second embodiment of the present disclosure;

FIG. 6 illustrates the optical performance (MTF) of the EDOF ophthalmic lens, according to a third embodiment of the present disclosure;

FIG. 7 illustrates the optical performance (MTF) of the EDOF ophthalmic lens, according to a fourth embodiment of the present disclosure;

FIG. 8 illustrates the optical performance (MTF) of the EDOF ophthalmic lens, according to a fifth embodiment of the present disclosure;

FIG. 9 illustrates the optical performance (MTF) of the EDOF ophthalmic lens, according to a sixth embodiment of the present disclosure;

FIG. 10 illustrates the optical performance (MTF) of the EDOF ophthalmic lens, according to a seventh embodiment of the present disclosure;

FIG. 11 shows a flowchart illustrating the design and manufacture of the double-sided aspheric ophthalmic lenses with phase-ring structures, according to some embodiments of the present disclosure;

FIG. 12A illustrates a top view of the EDOF ophthalmic lens, according to some embodiments of the present disclosure;

FIG. 12B illustrates a detailed view of the EDOF ophthalmic lens, according to some embodiments of the present disclosure;

FIG. 12C illustrates a cross-sectional view of the EDOF ophthalmic lens, according to some embodiments of the present disclosure;

FIG. 12D illustrates a cross-sectional view of a lens body of the EDOF ophthalmic lens, according to some embodiments of the present disclosure;

FIG. 13A illustrates a phase profile of the phase-ring structure of a multifocal ophthalmic lens, according to a first embodiment of the present disclosure;

FIG. 13B illustrates the phase profile of the phase-ring structure of the multifocal ophthalmic lens, according to a second embodiment of the present disclosure;

FIG. 14 illustrates the total height profile of an aspherical surface with a phase-ring structure of the multifocal ophthalmic lens, according to some embodiments of the present disclosure;

FIG. 15A illustrates an optical performance (modulation transfer function, MTF) of the multifocal ophthalmic lens, according to a first embodiment of the present disclosure;

FIG. 15B illustrates the optical performance (MTF) of the multifocal ophthalmic lens, according to a second embodiment of the present disclosure;

FIG. 15C illustrates the optical performance (MTF) of the multifocal ophthalmic lens, according to a third embodiment of the present disclosure;

FIG. 16A illustrates a top view of the multifocal ophthalmic lens, according to some embodiments of the present disclosure;

FIG. 16B illustrates a detailed view of the multifocal ophthalmic lens, according to some embodiments of the present disclosure;

FIG. 16C illustrates a cross-sectional view of the multifocal ophthalmic lens, according to some embodiments of the present disclosure; and

FIG. 16D illustrates a cross-sectional view of a lens body of the multifocal ophthalmic lens, according to some embodiments of the present disclosure.

### DETAILED DESCRIPTION

The present disclosure relates to double-sided aspheric ophthalmic lenses with phase-ring structures, such as extended depth of focus (EDOF) lenses and multifocal lenses, and methods of designing and manufacturing such lenses. The lenses can include a first aspheric (anterior) surface and a second aspheric (posterior) surface. One of the two surfaces can include a phase-ring structure, and the other surface can optionally include a toric component. The phase-ring structure may comprise a series of concentric regions extending from the center of the lens to the edge of the lens. The transition between regions in the phase-ring structure may be represented by a ring that corresponds to a ridge in the surface profile of the ophthalmic lens. The phase-ring structure is designed to provide the depth of focus via a non-diffracting beam principle with constructive phasing to maximize the focal energy. The multifocal and extended depth of focus lenses described herein may utilize refractive optics, such that the surface profiles of the phase-ring structures comprise sloped walls rather than, for example, vertical walls (or steps) associated with diffractive lens designs. Refractive lenses give focal points using the refractive effect of light waves at the refracting surface comprising boundary surfaces with different refractive indexes. Thus, when refraction of light passes through the one or more regions of the lenses described herein, constructive interference produces an extended depth of focus and improved distance focus, intermediate focus, and near focus.

The double-sided aspheric surface design results in an improvement of the modulation transfer function (MTF) of the lens-eye combination by aberration reduction and vision contrast enhancement as compared to a one-sided aspheric lens. The phase-ring structures described herein can enable an extended depth of focus for ophthalmic lenses, and as a set of lenses may produce continuous vision from a near focus, intermediate focus, and a distance focus. Various phase-ring structures are described in greater detail below with reference to two double-sided aspheric lens designs, hereinafter referred to as an extended depth of focus (EDOF) lens and a multifocal lens.

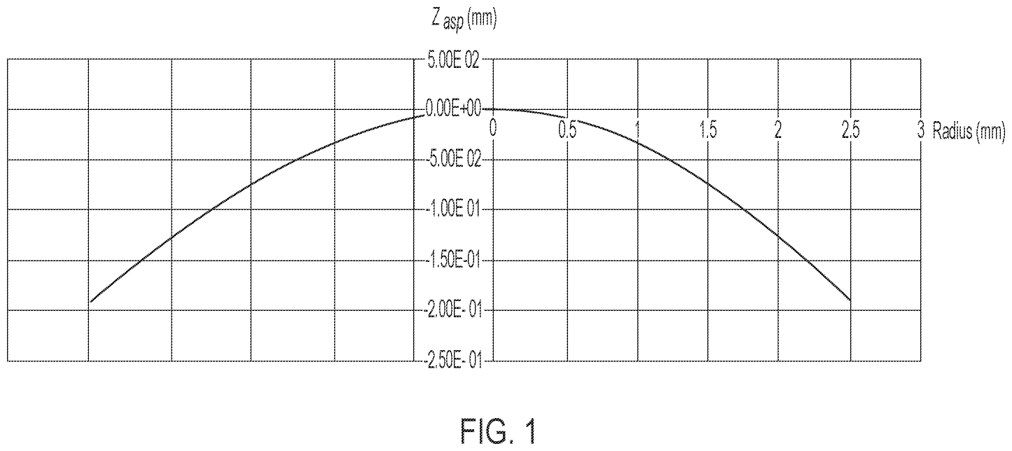

FIG. 1 illustrates the height profile of the aspherical base curvature as a function of the radial distance from the center of the ophthalmic lens, according to some embodiments of the present disclosure. The aspherical base curvature of FIG. 1 is based on the below parameters corresponding to the height profile expression provided in Equation (V) (described in greater detail below), where c is a curvature, k is a conic constant, and Aᵢ (where i is 4, 6, 8, etc.) is a higher order aspheric coefficient:

| c | k | A₄ | A₆ | A₈ |
|---|---|---|---|---|
| −0.06907 | 2.73263 | 0.00075054 | 6.89128E−06 | −2.81846E−08 |

The height profile illustrated in FIG. 1 may be applied to each of the lens designs described herein (e.g., the extended depth of focus lens and multifocal lens). The remaining figures provided herein may be described with respect to a specific lens design, denoted accordingly.

### Extended Depth of Focus (EDOF) Ophthalmic Lens

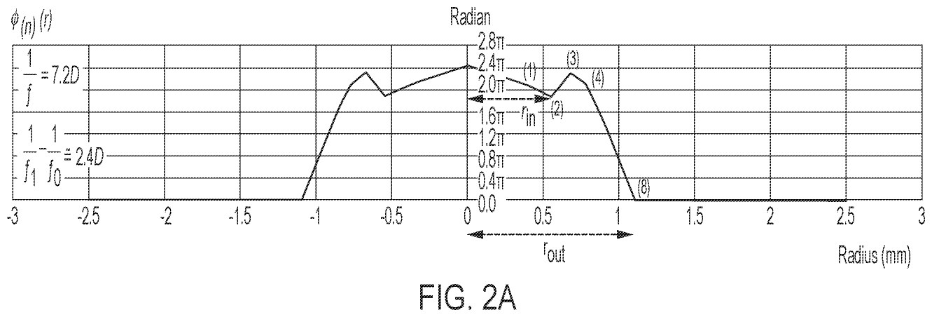

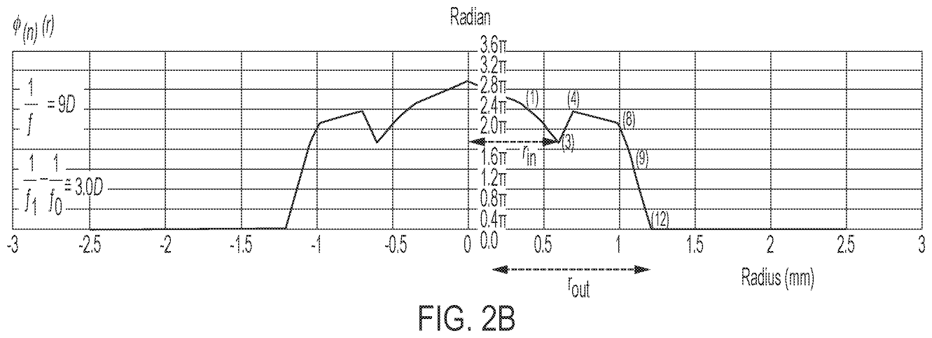

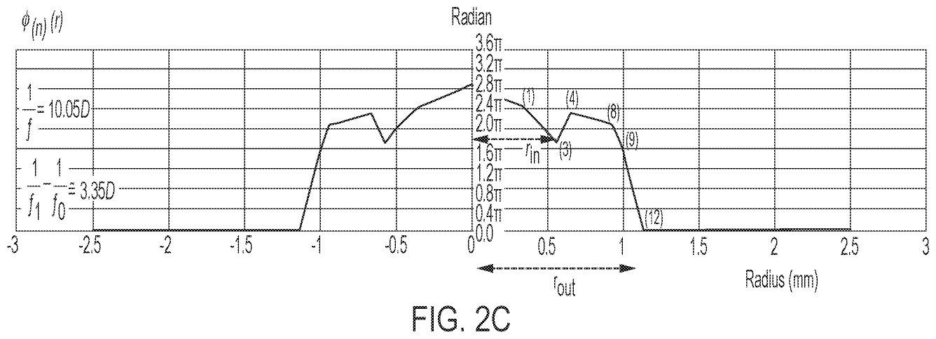

In some embodiments, a double-sided aspheric multifocal lens design with phase-ring structures may be provided for extending the depth of focus at least between an intermediate and far focus. FIGS. 2A-2C illustrate various phase profiles of the phase-ring structure of an extended depth of focus (EDOF) ophthalmic lens, according to some embodiments of the present disclosure. The phase profile of the phase-ring structure may correspond to the overall height profile of the phase-ring structure, as described below in relation to the height profile expression provided below in Equation (II). As mentioned above, the phase-ring structures of the lenses provided herein may comprise a series of concentric regions, such as 2, 3, 4 or more regions. FIGS. 2A-2C denote the respective regions in the corresponding phase profile diagram based on the radii of the regions.

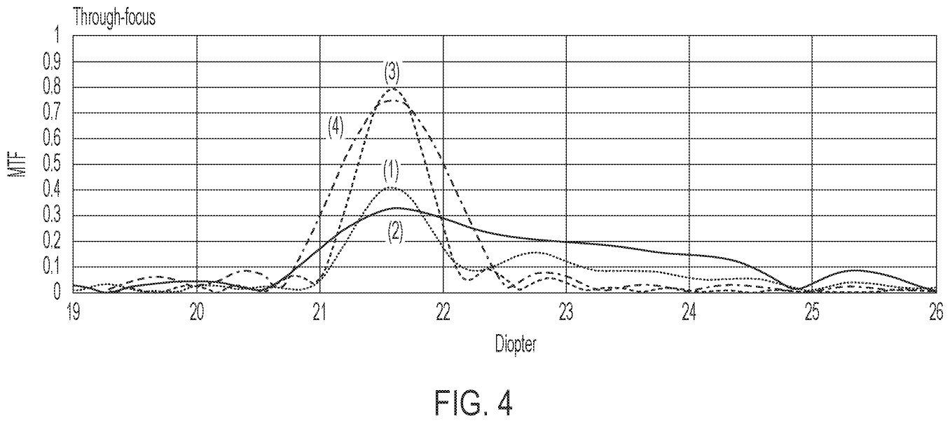

For example, FIG. 2A illustrates an inner radius (rᵢₙ) and outer radius (rₒᵤₜ) of a phase profile, the inner radius extending from an optical axis of the lens to an outer edge of an inner region of the phase-ring structure and the outer radius extending from the optical axis to an outer edge of an outer region of the phase-ring structure. The phase profile provided in FIG. 2A may correspond to the modulation transfer function (MTF) examples 1-3 illustrated in at least FIGS. 4-6, respectively, each of which are described in greater detail below with reference to the examples provided herein. The phase-ring structure illustrated in FIG. 2A may have a real add power of 2.4 D, the real add power expressed as the difference between the add power at a distance focus and the add power at an extended focus,

$$( \frac{1}{{f}_{1}} - \frac{1}{{f}_{0}} ) .$$

The real add power of a lens with a phase-ring structure corresponding to the phase profile of FIG. 2A may differ from the theoretical add power of a lens not having the phase-ring structure,

$$( \frac{1}{f} ) .$$

For example, the theoretical add power for a lens not having the phase-ring structure corresponding to the phase profile of FIG. 2A may be 7.2 D, which may be determined using equation (III) below. As mentioned above, a phase-ring structure can be designed to provide a depth-of-focus via a non-diffracting beam principle with constructive phasing to maximize the focal energy. Thus, when light passing through the lens refracts as it passes through the inner region and the outer region, constructive interference caused by refraction from the different regions in the EDOF lens may produce a distance focus and an extended focus.

In some embodiments, the inner region of the phase-ring structure may comprise a first phase-ring surface having a first curvature. The curvatures described herein may comprise one or more continuous arcs, parabolas, and/or lines. As shown, the first curvature may be monotonically decreasing (e.g., negative sloping) as it extends outwards from an optical axis of the lens. The first curvature may comprise a first (inner) sloping portion that slopes at a first angle with respect to an optical axis of the ophthalmic lens and a second (outer) sloping portion that slopes at a second angle with respect to the optical axis, each of the first and second sloping portions together sloping monotonically with respect to the optical axis. The first angle may be greater than the second angle such that the slope of the inner sloping portion may be less than that of the outer sloping portion. The inner radius (fin) may be greater than 0 mm and less than or equal to 5 mm, such as 0.30 mm, 0.35 mm, 0.40 mm, 0.45 mm, 0.55 mm, 0.60 mm, 0.65 mm, or 0.70 mm.

In some embodiments, the outer region of the phase-ring structure may comprise a second phase-ring surface having a second curvature. The second curvature may comprise a first (inner) sloping portion that slopes at a third angle with respect to the optical axis of the ophthalmic lens, a second (intermediate) sloping portion that slopes at a fourth angle with respect to the optical axis, and a third (outer) sloping portion that slopes at a fifth angle with respect to the optical axis. As shown, the second curvature may not be monotonically increasing or decreasing in a direction outwards from the optical axis of the lens. Rather, the intermediate sloping portion and the second outer sloping portion may together slope monotonically (e.g., both negative sloping) with respect to the optical axis, while the second inner sloping portion may slope in a direction opposite that of the intermediate sloping portion and the second outer sloping portion, such that the second curvature overall slopes non-monotonically. The fourth angle may be greater than the fifth angle such that the slope of the intermediate sloping portion may be less than that of the outer sloping portion. The outer radius (rₒᵤₜ) may be greater than 0 mm and less than or equal to 5 mm, such as 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, or 1.30 mm. The outer radius of the phase profile of the phase-ring structure may be proportional to the inner radius. For example, the relationship between the inner radius and outer radius may be expressed as 1≤rₒᵤₜ/rᵢₙ≤3.

In some embodiments, an outer edge of the inner region of the phase-ring structure may be adjacent to an inner edge of the outer region of the phase-ring structure. As shown in FIG. 2A, the transition between the first and second curvature may comprise a local minimum in the phase profile, at least because the sloping direction of the outer sloping portion of the first curvature (negative) may be the opposite of the sloping direction of the inner sloping portion of the second curvature (positive). Likewise, the second phase-ring surface may comprise a local maximum in the phase profile, at least because the sloping direction of the inner sloping portion (positive) and the intermediate sloping portion (negative) of the second curvature may be opposites. The local minimum and local maximum may correspond to locations of rings in the phase-ring structure. The region between a local minimum and a local maximum may in some embodiments be referred to as a transition region between two rings. In some embodiments, one or more rings in the phase-ring structure may be located between the rings corresponding to the local minimums and/or local maximums. Thus, the local minimums and maximums in the phase profile may lend to a ridged design in the profile of the lens surface profile, whereby at least the ridges in the surface profile may correspond to rings in the phase-ring structure.

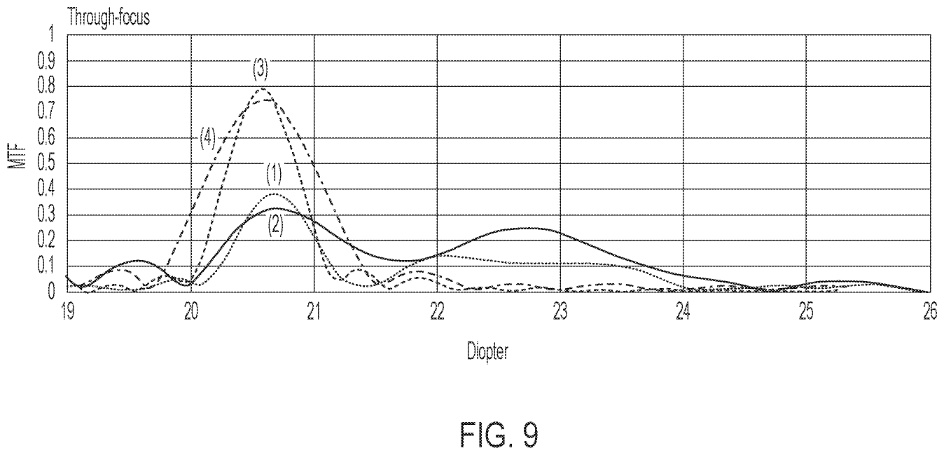

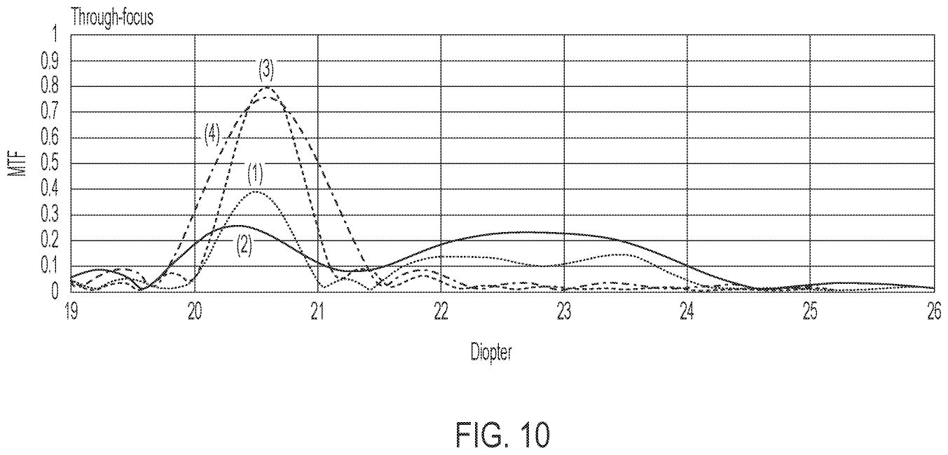

FIG. 2B illustrates a phase profile of a phase-ring structure of an EDOF ophthalmic lens, in accordance with some embodiments of the present disclosure. The phase profile illustrated in FIG. 2B may comprise one or more features of the phase profile described above with respect to FIG. 2A. For example, the phase-ring structure corresponding to the phase profile illustrated in FIG. 2B may comprise an inner region and an outer region, whereby the inner radius (rᵢₙ) may extend from an optical axis of the lens to an outer edge of the inner region of the phase-ring structure, and the outer radius (rₒᵤₜ) may extend from the optical axis to an outer edge of the outer region of the phase-ring structure. The inner region may comprise a first phase-ring surface having a first curvature and the outer region may comprise a second phase-ring surface having a second curvature. The phase profile provided in FIG. 2B may correspond to the modulation transfer function (MTF) examples 6-7 illustrated in at least FIGS. 9-10, respectively, each of which are described in greater detail below. The phase-ring structure illustrated in FIG. 2B may have a real add power

$$( \frac{1}{{f}_{1}} - \frac{1}{{f}_{0}} )$$

of 3.0 D. The theorical add power for a lens not having the phase-ring structure corresponding to the phase profile of FIG. 2B may be 9 D.

The inner radius (rᵢₙ) of the phase profile in FIG. 2B may be greater than 0 mm and less than or equal to 5 mm, such as 0.30 mm, 0.35 mm, 0.40 mm, 0.45 mm, 0.55 mm, 0.60 mm, 0.65 mm, or 0.70 mm. The outer radius (rₒᵤₜ) may be greater than 0 mm and less than or equal to 5 mm, such as 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, or 1.30 mm. The outer radius of the phase profile of the phase-ring structure may be proportional to the inner radius. For example, the relationship between the inner radius and outer radius may be expressed as 1≤rₒᵤₜ/rᵢₙ≤3.

The phase profile (Φ₍ₙ₎(r)) of the phase-ring structures corresponding to the schematics provided in FIGS. 2A-2C may be between 0 and 8π. Stated otherwise, 0≤|Φ₍ₙ₎(r)|≤8π. The height ratio between the inner region and the outer region of the phase-ring structure corresponding to the phase profiles in FIG. 2A-2C may be between 0 and 4π, otherwise expressed as 0≤Φ₍ₙ₎(rₒᵤₜ)−Φ₍ₙ₎(rᵢₙ)≤4π.

In some embodiments, the phase profile illustrated in FIG. 2B may differ from that illustrated in FIG. 2A at least because the height profile of the inner region may be less than that of the outer region in the phase profile illustrated in FIG. 2A, whereas the height profile of the inner region may be greater than that of the outer region in the phase profile illustrated in FIG. 2B.

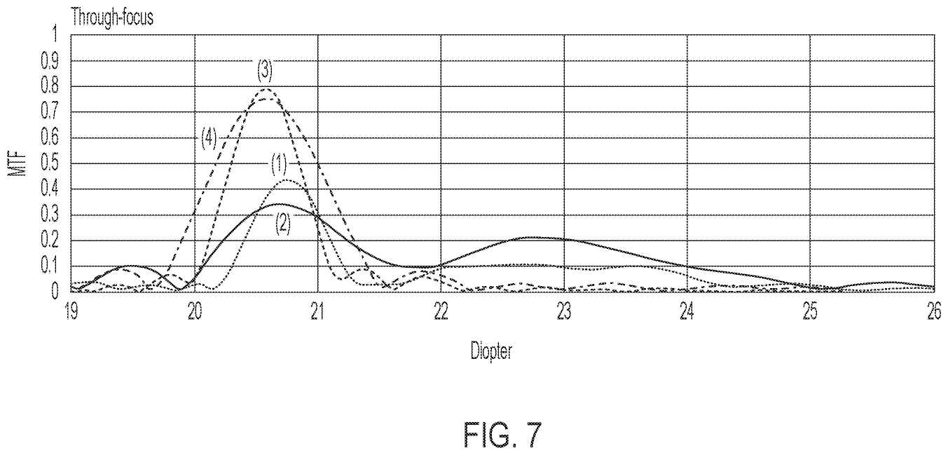

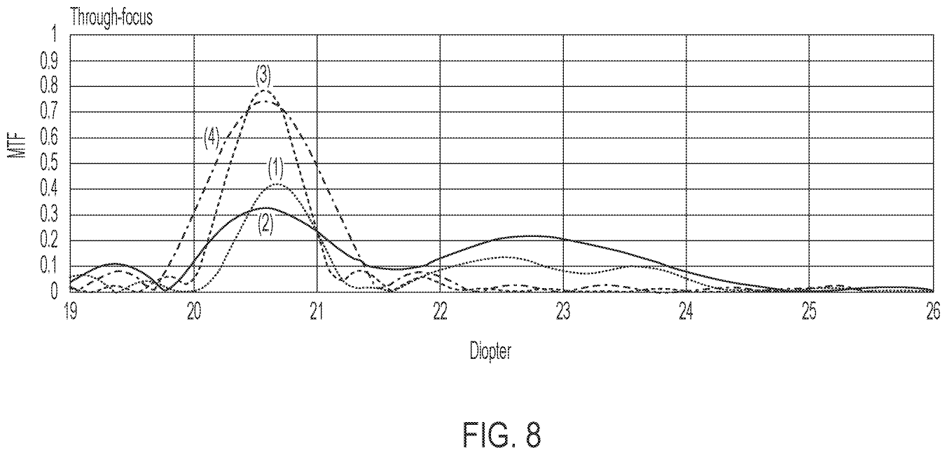

FIG. 2C illustrates a phase profile of a phase-ring structure of an EDOF ophthalmic lens, in accordance with some embodiments of the present disclosure. The phase profile illustrated in FIG. 2C may comprise one or more features of the phase profiles described above with respect to FIGS. 2A-2B. For example, the phase-ring structure corresponding to the phase-profile illustrated in FIG. 2C may comprise an inner region and an outer region, whereby the inner radius (rᵢₙ) may extend from an optical axis of the lens to an outer edge of the inner region of the phase-ring structure, and the outer radius (rₒᵤₜ) may extend from an optical axis to an outer edge of the outer region of the phase-ring structure. The inner region may comprise a first phase-ring surface having a first curvature and the outer region may comprise a second phase-ring surface having a second curvature. The phase profile provided in FIG. 2C may correspond to the modulation transfer function (MTF) examples 4-5 illustrated in at least FIGS. 7-8, respectively, each of which are described in greater detail below. The phase-ring structure illustrated in FIG. 2C may have a real add power

$$( \frac{1}{{f}_{1}} - \frac{1}{{f}_{0}} )$$

of 3.35 D. The theoretical add power for a lens not having the phase-ring structure corresponding to the phase profile of FIG. 2C may be 10.05 D.

The inner radius (rᵢₙ) of the phase profile in FIG. 2C may be greater than 0 mm and less than or equal to 5 mm, such as 0.30 mm, 0.35 mm, 0.40 mm, 0.45 mm, 0.55 mm, 0.60 mm, 0.65 mm, or 0.70 mm. The outer radius (rₒᵤₜ) may be greater than 0 mm and less than or equal to 5 mm, such as 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, or 1.30 mm. The outer radius of the phase profile of the phase-ring structure may be proportional to the inner radius. For example, the relationship between the inner radius and outer radius may be expressed as 1≤rₒᵤₜ/rᵢₙ≤3.

In some embodiments, the phase profile illustrated in FIG. 2C may differ from that illustrated in FIG. 2A at least because the height profile of the inner region may be less than that of the outer region in the phase profile illustrated in FIG. 2A, whereas the height profile of the inner region may be greater than that of the outer region in the phase profile illustrated in FIG. 2C.

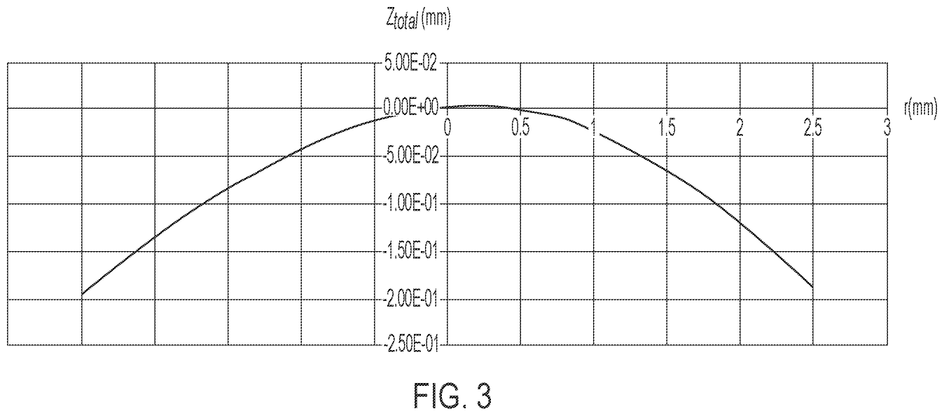

FIG. 3 illustrates the height profile of the combined aspherical base curve with the phase-ring structure, according to some embodiments of the present disclosure. As described in greater detail below (with respect at least to Equation (VI)), the height profile of the combined structure may be the summation of the height profile of the aspheric base curvature and the height profile of the phase-ring structure.

FIGS. 4-10 illustrate the optical performance (MTF) of various extended depth of focus (EDOF) ophthalmic lenses with phase-ring structures, in accordance with some embodiments of the present disclosure. Each of FIGS. 4-10 will be described in greater detail below in the provided examples section.

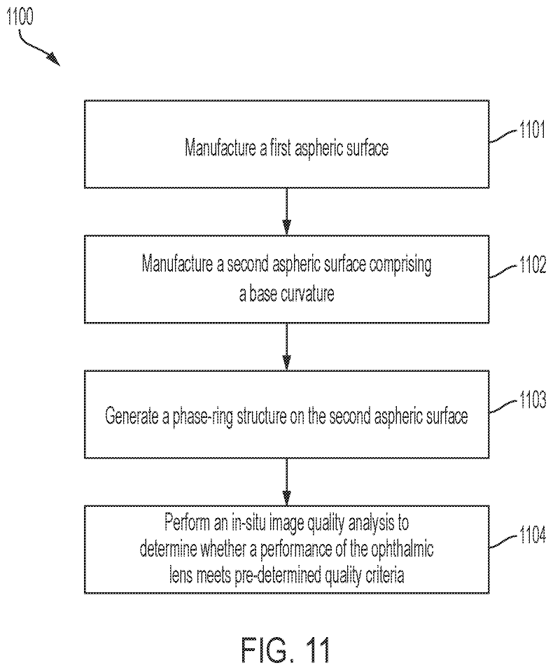

FIG. 11 shows a flowchart 1100 illustrating the manufacture of the double-sided aspheric ophthalmic lenses with phase-ring structures, according to some embodiments of the present disclosure. For example, the method of flowchart 1100 may be used to manufacture the above-described EDOF lens and/or the below-described multifocal lens. At step 1101, a first aspheric surface may be manufactured. At step 1102 a second aspheric surface may be manufactured comprising a base curvature. At step 1103, a phase-ring structure may be generated on the second aspheric surface, the phase-ring structure comprising an inner region and an outer region. For example, the phase-ring structure may be generated by cutting the aspheric surface using a lathe that may be equipped with a cutting head made of a hard mineral such as diamond or sapphire; direct write patterning using a high energy beam such as a laser beam or electron beam or a similar method of ablating the surface; etching the surface using a photolithographic patterning process; or molding the surface.

The inner region may comprise a first phase-ring surface having a first curvature and the outer region may comprise a second phase-ring surface having a second curvature. An outer edge of the inner region may be adjacent to an inner edge of the outer region. The manufactured ophthalmic lens may produce a distance focus and an extended focus. Optionally, at step 1104 an in-situ image quality analysis of the double-sided aspheric phase-ring structured lens may be performed. For example, an ISO Model Eye 2 may be used to measure the through-focus MTF using the TRIOPTICS OptiSpheric® IOL PRO 2 to determine whether a performance of the ophthalmic lens meets predetermined quality criteria.

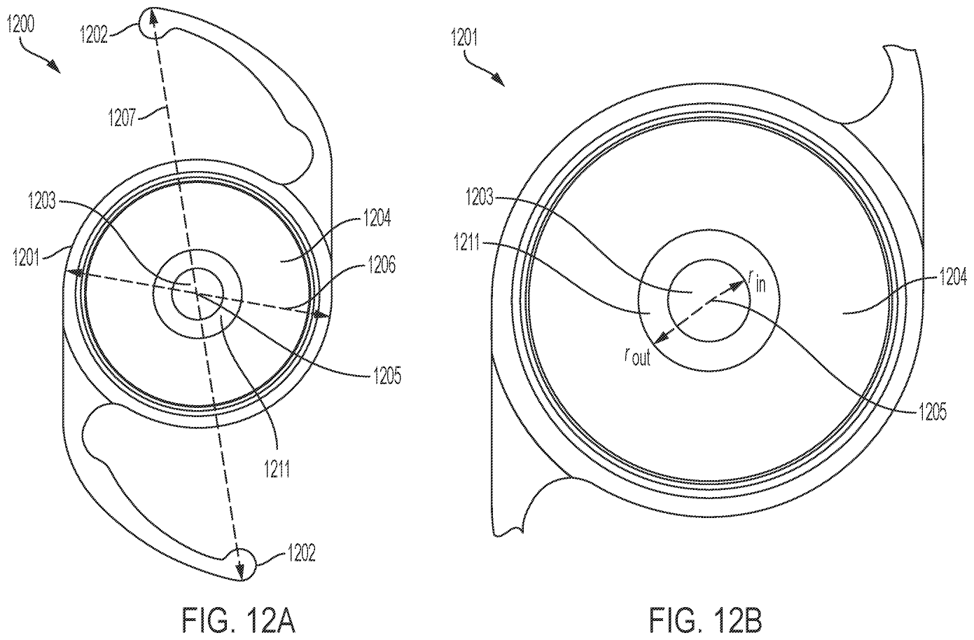

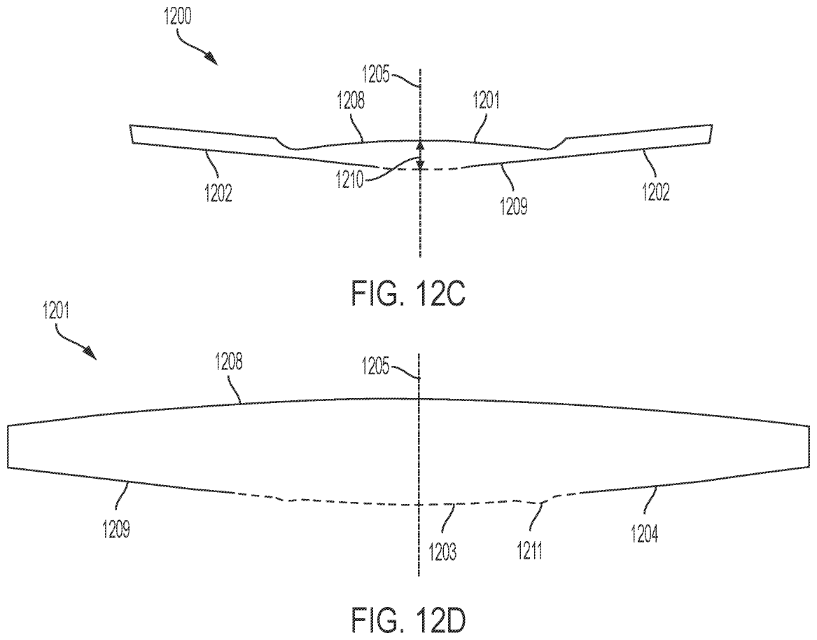

FIGS. 12A-12D illustrate a double-sided aspheric phase-ring structured ophthalmic lens with an extended depth of focus (EDOF), according to some embodiments of the present disclosure. FIG. 12A shows a top view of an EDOF ophthalmic lens 1200, FIG. 12B shows a detailed view of the phase-ring structure of the EDOF ophthalmic lens 1200, FIG. 12C shows cross-sectional view of the EDOF ophthalmic lens 1200, and FIG. 12D shows a cross-sectional view of a lens body of the EDOF ophthalmic lens 1200.

Lens 1200 can include a light transmissive circular disk-shaped lens body 1201 with an optic diameter 1206 and a center thickness 1210, as well as a pair of haptics 1202 as flexible support for the IOL when implanted into patient's eye, with a total outer diameter 1207. Lens body 1201 can include an anterior surface 1208, a posterior surface 1209, a central zone 1203 and a surrounding area 1204. As mentioned above, lens body 1201 can include an optical axis 1205 extending transverse to the anterior surface 1208 and posterior surface 1209. A person of skill in the art will appreciate that the optical axis 1205 is a virtual axis for purposes of referring to the optical properties of lens 1200. The pair of haptics 1202 can be extended outwardly from the lens body 1201 for supporting the lens 1200 after being implanted in the human eye. In some embodiments, the haptics 1202 of lens 1200 can hold the lens in place in the capsular bag.

In some embodiments, lens body 1201 can take the shape of a biconvex shape. Other shapes of lens body 1201 can include but are not limited to, plano-convex, biconcave, plano-concave shape, or combinations of convex and concave shapes. In some embodiments, both anterior surface 1208 and posterior surface 1209 can feature an aspheric structure, providing a double-sided asphericity for lens 1200.

As mentioned above at least with respect to FIGS. 2A-2C, lens 1200 can include a phase-ring structure comprising one or more regions. The phase-ring structure can be designed to provide a depth-of-focus via a non-diffracting beam principle with constructive phasing to maximize the focal energy. In some embodiments, the phase-ring structure of lens 1200 can include 1, 2, 3, 4, 5 or more regions. For example, lens 1200 illustrated at least in FIGS. 12A-12B may include an inner region (otherwise referred to herein as central zone 1203) and outer region 1211. Lens 1200 may include phase-ring structures on one of the surfaces or both surfaces of the lens. In some embodiments, the phase-ring structures can be placed on the posterior surface of the lens. In some embodiments, the phase-ring structures can be placed at the posterior surface because there is less light scattering effect at the posterior surface than at the anterior surface. As mentioned above, each of the regions may comprise and/or be bounded by one or more rings extending concentrically with respect to an optical axis through the central zone 1203 (or inner region) over at least part of the posterior surface of the lens body 1201. In some embodiments, the regions may not be limited to concentric circular or annular ring-shaped regions but may instead or additionally include concentric elliptic or oval-shaped regions.

In some embodiments, the optic diameter 1206 of lens body 1201 may be greater than or equal to about 4 mm, such as about 4.5 mm, 5 mm, 5.5 mm, 6 mm, 6.5 mm, 7 mm, 7.5 mm, or 8 mm. The total outer diameter 1207 of lens 1200 including the haptics 1202 may be greater than or equal to about 9 mm, such as 10 mm, 11 mm, 12 mm, 13 mm, 14 mm, 15 mm, 16 mm, 17 mm, or 18 mm. Lens body 1201 may have a center thickness 1210 greater than or equal to about 0.8 mm, such as 0.85 mm, 0.9 mm, 0.95 mm, 1.0 mm, 1.05 mm, 1.10 mm, 1.15 mm, or 1.20 mm. One of ordinary skill in the art will recognize that although FIGS. 12A-12B illustrate an intraocular lens (IOL), other ophthalmic lenses, including multifocal diffractive contact lenses or eye glass lenses, could also benefit from the same approach. When used for ophthalmic multifocal contact lenses and spectacle or eye glass lenses, haptics 1202 may not be provided.

The amount of correction that an ophthalmic lens provides is called optical power and is expressed in Diopter (D). The optical power is calculated as the inverse of a focal distance f measured in meters, which can be a respective focal distance from the lens to a respective focal point for far, intermediate, or near vision. Lens body 1201 (and lens body 1601 described in greater detail below) can provide a base optical power of about −15 D to about +55 D.

The ophthalmic lenses of the present disclosure, such as lens 1200 (and lens 1600 described in greater detail below), can be made of flexible material which permits a reduction of their overall apparent girth by temporary deformation, facilitating their insertion through the cornea, thereby advantageously enabling the use of a corneal incision of concomitantly reduced size. In some embodiments, the lens body can include polypropylene, polycarbonate, polyethylene, acryl-butadiene styrene, polyamide, polychlorotrifluoroethylene, polytetrafluoroethylene, polyvinyl chloride, polyvinylidene fluoride, polyvinylchloride, polydimethylsiloxane, polyethylene terephthalate, ethylene tetrafluoroethylene, ethylene chlorotrifluoroethylene, perfluoroalkoxy, polymethylpentene, polymethylmethacrylate, polystyrene, polyetheretherketone, tetrafluoroethylene, polyurethane, poly(methyl methacrylate), poly(2-hydroxyethyl methacrylate), nylon, polyether block amide, silicone or a mixture thereof.

In some embodiments, the lens body can include a hydrophilic polymer made of monomers selected from the group consisting of: 2-acrylamido-2-methylpropane sulfonic acid, 2-hydroxyethyl methacrylate, N-vinylpyrrolidone, vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary butylaminoethyl acrylate, tertiary-butylaminoethyl methacrylate and dimethylaminopropylacrylamide, acrylic acid, methacrylic acid, styrenesulfonic acid and salts thereof, hydroxypropyl acrylate, vinylpyrrolidone, dimethylacrylamide, ethylene glycol monomethacrylate, ethylene glycol monoacrylate, ethylene glycol dimethacrylate, ethylene glycol diacrylate, triethylene glycol diacrylate and triethylene glycol methacrylate. In some embodiments, these hydrophilic monomers are surface grafted onto the polymeric matrix mentioned above to make the lens body. In some embodiments, the ophthalmic lenses of the present disclosure can be made of polymeric compositions according to U.S. Pat. No. 10,494,458, titled “Functionalized hydrophilic and lubricious polymeric matrix and methods of using same,” which is incorporated herein by reference in its entirety.

As mentioned above, the ophthalmic lens of the present disclosure can be an intraocular lens (IOL). The haptics of the IOL according to the present disclosure can be made of polymeric materials including but not limited to polymethacrylate, polypropylene, polyethylene, polystyrene, and polyacrylate.

The surface of the IOL can include spheric, aspheric, and/or toric elements. Spheric surfaces can cause spherical aberration, which is a type of optical imperfection that can cause increased glare and reduced overall quality of vision especially in low light and darkness. Aspheric lenses can correct spherical aberration. Aspherical IOLs can provide improved contrast sensitivity, enhanced functional vision and superior night driving ability.

A toric element is typically used for astigmatic eye correction. Generally, astigmatism is an optical defect in which vision is blurred due to the ocular inability to focus a point object into a sharply focused image on the retina. This inability may be due to an irregular curvature of the cornea and/or lens. The refractive error of the astigmatic eye stems from a difference in degree of curvature, and therefore in degree of refraction, of the different meridians of the cornea and/or the crystalline lens, which causes the eye to have two focal points, one correspondent to each meridian. As used herein, a meridian includes one of two axes that subtend a curved surface, such as the prime meridian on the earth, for example. Meridians may be orthogonal. By way of example, the meridians of the earth may be any orthogonal line of longitude and any line of latitude that curve about the surface of the earth.

For example, in an astigmatic eye, an image may be clearly focused on the retina in the horizontal (sagittal) plane but may be focused behind the retina in the vertical (tangential) plane. In the case where the astigmatism results only from the cornea, the two astigmatism meridians may be the two axes of the cornea. If the astigmatism results from the crystalline lens, the two astigmatism meridians may be the two axes of the crystalline lens. If the astigmatism results from a combination of the cornea and the crystalline lens, the two astigmatism meridians may be the respective axes of the combined lenses of the cornea and the crystalline lens.

An astigmatism arising from the cornea or crystalline lens, or the combination of the two lenses, may be corrected by a lens including a toric component. A toric surface resembles a section of the surface of a football, for which there are two regular radii of curvature, one smaller than another. These radii may be used to correct the defocus in the two meridians of the astigmatic eye. Thus, blurred vision caused by astigmatism may be corrected by corrective lenses or laser vision correction, such as glasses, hard contact lenses, contact lenses, and/or intraocular lenses (IOLs), providing a compensating optic specifically rotated around the optical axis.

In some embodiments, the ophthalmic lenses according to the present disclosure can provide far vision for viewing objects at distances ranging from about infinity to about 6 meters (m). In some embodiments, one or more lenses of the present disclosure can provide near vision for viewing objects at distances less than about 3 m. In some embodiments, the lenses of the present disclosure can provide intermediate vision for viewing objects at distances in a range of about 0.3 m to about 1 m, about 2 m, about 3 m, about 4 m, about 5 m, or about 6 m. As a result, the lens of the present disclosure can advantageously provide a degree of accommodation for different distance ranges, typically referred to as pseudo-accommodation. In some embodiments, when implanted into a patient's eye, the combined power of the eye's cornea and the near, intermediate, and far power of the ophthalmic lens of the present disclosure can allow focusing light emanating from objects within a near, an intermediate, and a far distance range of the patient onto the retina. In some embodiments, the distance focus (f₀) and extended focus (f₁) provided by the IOLs of the present disclosure can be defined by the following expressions:

$$- 15 D \leq \frac{1}{{f}_{0}} \leq 55 D , \frac{1}{6 f} \leq \frac{1}{{f}_{1}} - \frac{1}{{f}_{0}} \leq \frac{1}{2 f} , and 3 D \leq \frac{1}{f} \leq 16 D ,$$

wherein

$$\frac{1}{f}$$

is representative of the theoretical add power, and

$$\frac{1}{{f}_{1}} - \frac{1}{{f}_{0}}$$

is representative of the real add power.

### Multifocal Ophthalmic Lens

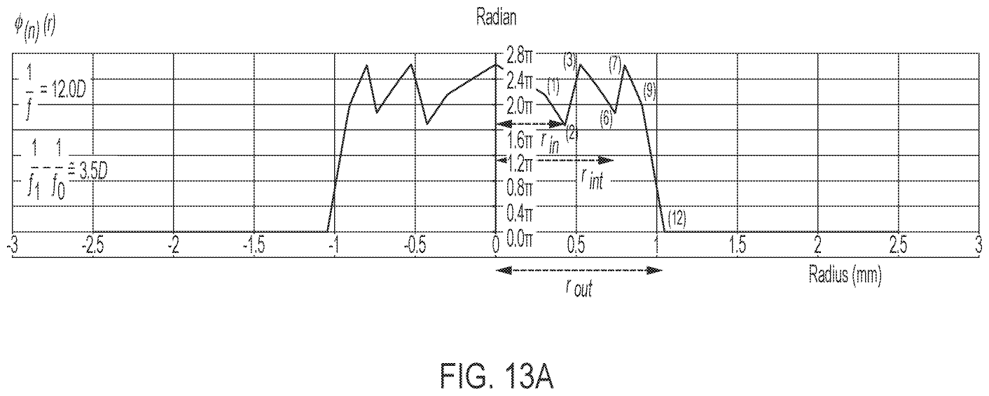

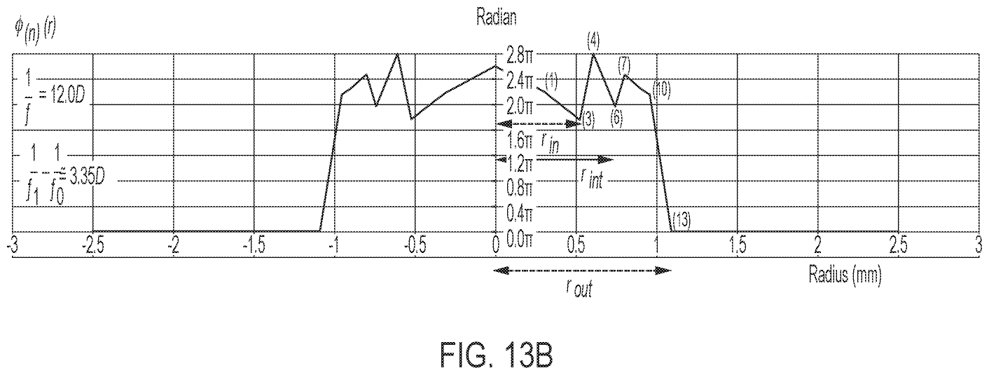

In some embodiments, a double-sided aspheric multifocal lens design with phase-ring structures may be provided for improving far and near vision. The multifocal lens may minimize undesirable visual effects, such as dysphotopsia (glare, halos, etc.) experienced during use of ophthalmic lenses, and in specific, during nighttime use. FIGS. 13A-13B illustrate various phase profiles of the phase-ring structure of a multifocal ophthalmic lens, according to some embodiments of the present disclosure. The phase profile of the phase-ring structure may correspond to the overall height profile of the phase-ring structure, as described below in relation to the height profile expression provided below in Equation (II). As mentioned above, the phase-ring structures of the lenses provided herein may comprise a series of concentric regions, such as 2, 3, 4 or more regions. FIGS. 13A-13B denote the respective regions in the corresponding phase profile diagram based on the radii of the regions.

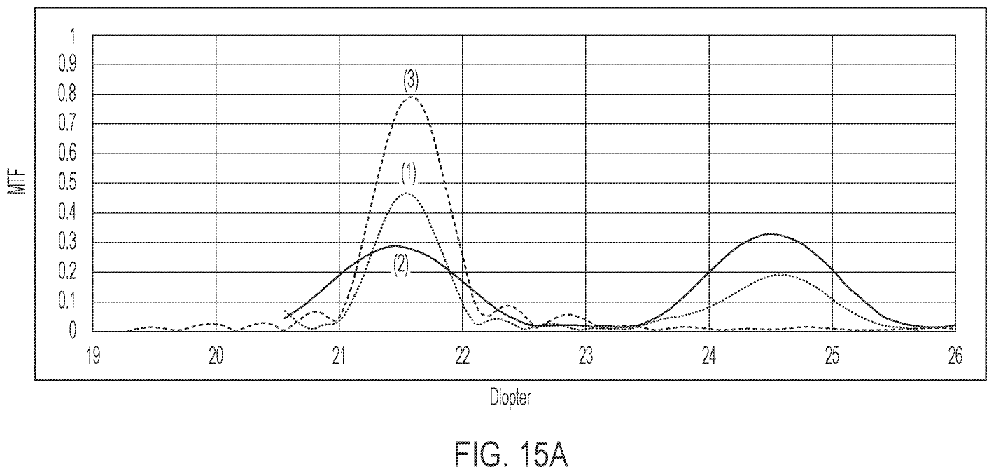

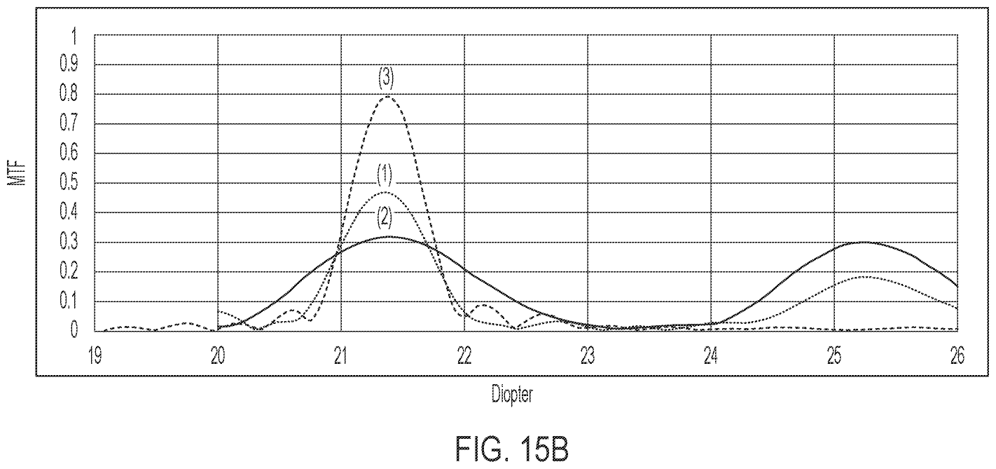

For example, FIG. 13A illustrates an inner radius (rᵢₙ), intermediate radius (rᵢₙₜ), and outer radius (rₒᵤₜ) of a phase profile, the inner radius extending from an optical axis of the lens to an outer edge of an inner region of the phase-ring structure, the intermediate radius extending from the optical axis to an outer edge of the intermediate region of the phase-ring structure, and the outer radius extending from the optical axis to an outer edge of an outer region of the phase-ring structure. The phase profile provided in FIG. 13A may correspond to the modulation transfer function (MTF) examples 8-9, respectively illustrated in FIGS. 15A-15B, each of which are described in greater detail below with reference to the examples provided herein. The phase-ring structure illustrated in FIG. 13A may have a real add power of 3.5 D, the real add power expressed as the difference between the add power at a distance focus and the add power at an extended focus

$$( \frac{1}{{f}_{1}} - \frac{1}{{f}_{0}} ) .$$

The real add power of a lens with a phase-ring structure corresponding to the phase profile of FIG. 13A may differ from the theoretical add power of a lens not having the phase-ring structure,

$$( \frac{1}{f} ) .$$

For example, the theoretical add power for a lens not having the phase-ring structure corresponding to the phase profile of FIG. 13A may be 12.0 D, which may be determined using equation (III) below. As mentioned above, a phase-ring structure can be designed to provide a depth-of-focus via a non-diffracting beam principle with constructive phasing to maximize the focal energy. Thus, when refraction of light passes through the inner region, intermediate region, and the outer region, constructive interference in the multifocal lens may produce a near focus, intermediate focus, and a distance focus.

In some embodiments, the inner region of the phase-ring structure may comprise a first phase-ring surface having a first curvature. The curvatures described herein may comprise one or more continuous arcs, parabolas, and/or lines. As shown, the first curvature may be monotonically decreasing (e.g., negative sloping) as it extends outwards from an optical axis of the lens. The first curvature may comprise a first (inner) sloping portion that slopes at a first angle with respect to an optical axis of the ophthalmic lens and a second (outer) sloping portion that slopes at a second angle with respect to the optical axis. The first sloping portion and the second sloping portion together may slope monotonically with respect to the optical axis of the ophthalmic lens. The first angle may be greater than the second angle such that the slope of the inner sloping portion may be less than that of the outer sloping portion. The inner radius (rᵢₙ) may be greater than 0 mm and less than or equal to 5 mm, such as 0.30 mm, 0.35 mm, 0.40 mm, 0.45 mm, 0.55 mm, 0.60 mm, 0.65 mm, or 0.70 mm.

In some embodiments, the intermediate region of the phase-ring structure may comprise a second phase-ring surface having a second curvature. The second curvature may comprise a first (inner) sloping portion that slopes at a third angle with respect to the optical axis of the ophthalmic lens and a second (outer) sloping portion that slopes at a fourth angle with respect to the optical axis. As shown, the second curvature may not be monotonically increasing or decreasing in a direction extending outwards from the optical axis of the lens. Rather, the inner sloping portion and the outer sloping portion may slope in different directions. The intermediate radius (rᵢₙₜ) may be greater than 0 mm and less than or equal to 5 mm, such as 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, or 1.30 mm.

In some embodiments, the outer region of the phase-ring structure may comprise a third phase-ring surface having a third curvature. The third curvature may comprise a first (inner) sloping portion that slopes at a fifth angle with respect to the optical axis of the ophthalmic lens and a second (outer) sloping portion that slopes at a sixth angle with respect to the optical axis. As shown, the third curvature may not be monotonically increasing or decreasing in a direction outwards from the optical axis of the lens. Rather, the inner sloping portion and the outer sloping portion may slope in different directions. The outer radius (rₒᵤₜ) may be greater than 0 mm and less than or equal to 5 mm, such as 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, or 1.30 mm. The outer radius of the phase profile of the phase-ring structure may be proportional to the inner radius. For example, the relationship between the inner radius and outer radius may be expressed as 1≤rₒᵤₜ/rᵢₙ≤3.

In some embodiments, an outer edge of the inner region of the phase-ring structure may be adjacent to an inner edge of the intermediate region of the phase-ring structure. Likewise, an outer edge of the intermediate region may be adjacent to an inner edge of the outer region of the phase-ring structure. As shown in FIG. 13A, the transition between the first and second curvature may comprise a local minimum in the phase profile, at least because the sloping direction of the outer sloping portion of the first curvature (negative) may be the opposite of the sloping direction of the inner sloping portion of the second curvature (positive). Likewise, the transition between the second and third curvature may comprise a local minimum in the phase profile, at least because the sloping direction of the outer sloping portion of the second curvature may be the opposite of the sloping direction of the inner sloping portion of the third curvature. The second phase-ring surface and third phase-ring surface may comprise local maximums in the phase profile, at least because the sloping direction of the inner sloping portion (positive) and the outer sloping portion (negative) of each of the second curvature and third curvature may be opposites. The local minimums and local maximums may correspond to locations of rings in the phase-ring structure. The region between a local minimum and a local maximum may in some embodiments be referred to as a transition region between two rings. In some embodiments, one or more rings in the phase-ring structure may be located between the rings corresponding to the local minimums and/or local maximums. Thus, the local minimums and maximums in the phase profile may lend to a ridged design in the profile of the lens surface profile, whereby at least the ridges in the surface profile may correspond to rings in the phase-ring structure.

FIG. 13B illustrates a phase profile of a phase-ring structure of a multifocal ophthalmic lens, in accordance with some embodiments of the present disclosure. The phase profile illustrated in FIG. 13B may comprise one or more features of the phase profile described above with respect to FIG. 13A. For example, the phase-ring structure corresponding to the phase profile illustrated in FIG. 13B may comprise an inner region, intermediate region, and an outer region, whereby the inner radius (rᵢₙ) may extend from an optical axis of the lens to an outer edge of the inner region of the phase-ring structure, the intermediate radius (rᵢₙₜ) may extend from the optical axis to an outer edge of the intermediate region of the phase-ring structure, and the outer radius (rₒᵤₜ) may extend from the optical axis to an outer edge of the outer region of the phase-ring structure. The inner region may comprise a first phase-ring surface having a first curvature, the intermediate region may comprise a second phase-ring surface having a second curvature, and the outer region may comprise a third phase-ring surface having a third curvature. The phase profile provided in FIG. 13B may correspond to the modulation transfer function (MTF) example 12 illustrated in FIG. 15C, which is described in greater detail below. The phase-ring structure illustrated in FIG. 13B may have a real add power

$$( \frac{1}{{f}_{1}} - \frac{1}{{f}_{0}} )$$

of 3.35 D. The theoretical add power for a lens not having the phase-ring structure corresponding to the phase profile of FIG. 13B may be 12 D.

The inner radius (rᵢₙ) of the phase profile in FIG. 13B may be greater than 0 mm and less than or equal to 5 mm, such as 0.30 mm, 0.35 mm, 0.40 mm, 0.45 mm, 0.55 mm, 0.60 mm, 0.65 mm, or 0.70 mm. The intermediate radius (rᵢₙₜ) may be greater than 0 mm and less than or equal to 5 mm, such as 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, or 1.30 mm. The outer radius (rₒᵤₜ) may be greater than 0 mm and less than or equal to 5 mm, such as 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, or 1.30 mm. The outer radius of the phase profile of the phase-ring structure may be proportional to the inner radius.

The phase profile (Φ₍ₙ₎(r)) of the phase-ring structures corresponding to the schematics provided in FIGS. 13A-13B may be between 0 and 8π. Stated otherwise, 0≤|Φ₍ₙ₎(r)|≤8π. The height ratio between the inner region and the intermediate region, the inner region and the outer region, and/or the intermediate region and the outer region of the phase-ring structure corresponding to the phase profiles in FIG. 13A-13B may be between 0 and 4π, otherwise expressed as 0≤Φ₍ₙ₎(rₒᵤₜ)−Φ₍ₙ₎(rᵢₙ)≤4π.

In some embodiments, the phase profile illustrated in FIG. 13B may differ from that illustrated in FIG. 13A at least because the height profile of the inner region may be less than that of the intermediate and/or outer region in the phase profile illustrated in FIG. 13B, whereas the height profile of the inner region may be substantially the same as that of the intermediate region and/or outer region in the phase profile illustrated in FIG. 13A.

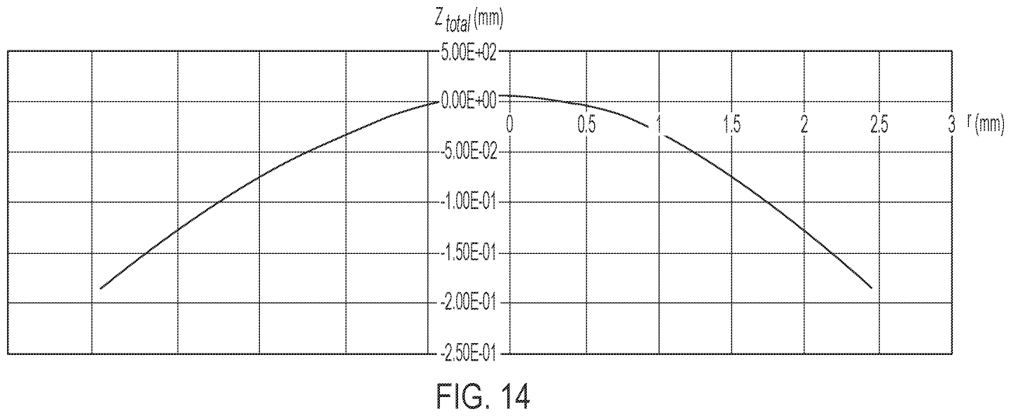

FIG. 14 illustrates the height profile of the combined aspherical base curve with the phase-ring structure for the multifocal lens, according to some embodiments of the present disclosure. As described in greater detail below (Equation (VI)), the height profile of the combined structure may be the summation of the height profile of the aspheric base curvature and the height profile of the phase-ring structure.

FIGS. 15A-15C illustrate the optical performance (MTF) of various multifocal ophthalmic lenses with phase-ring structures, in accordance with some embodiments of the present disclosure. Each of FIGS. 15A-15C will be described in greater detail below in the provided examples section.

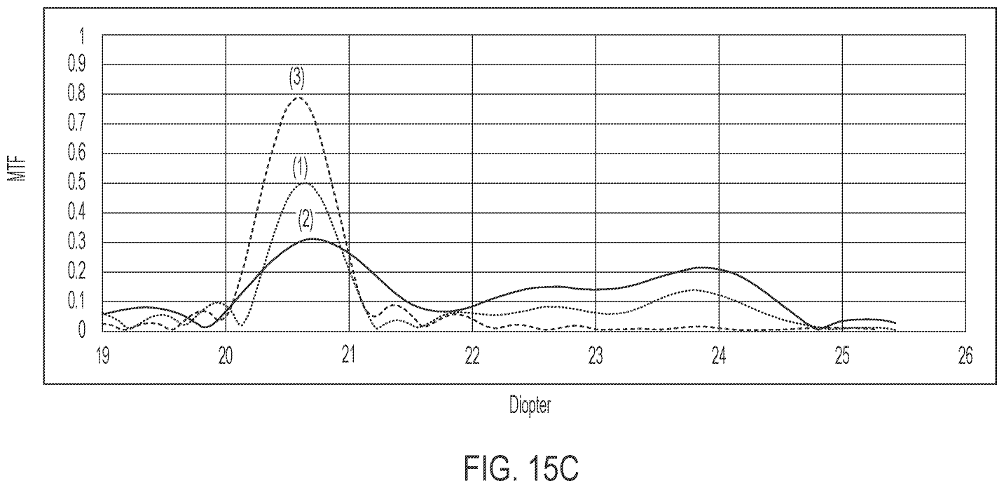

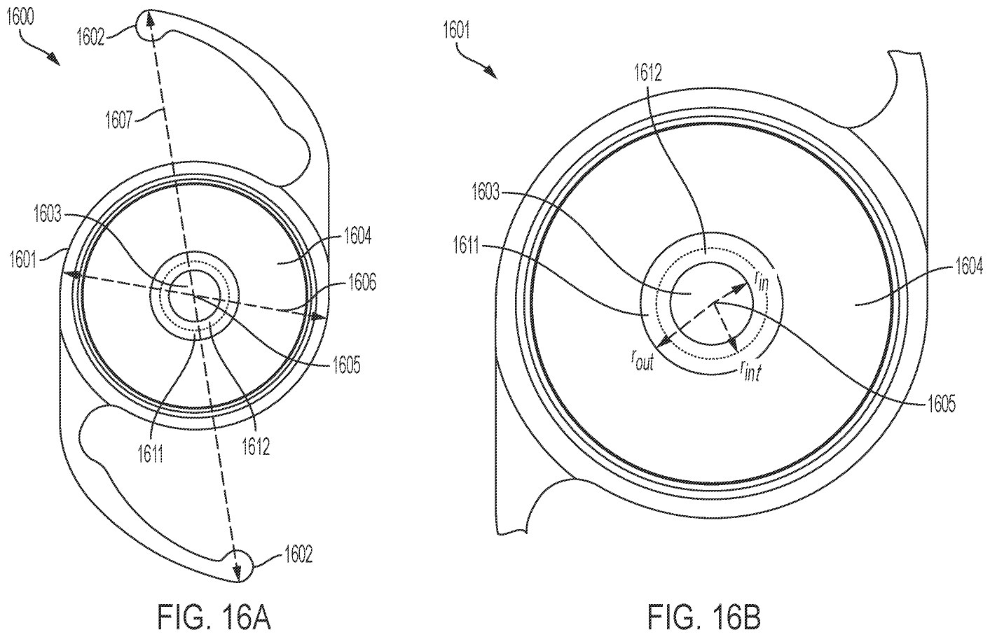

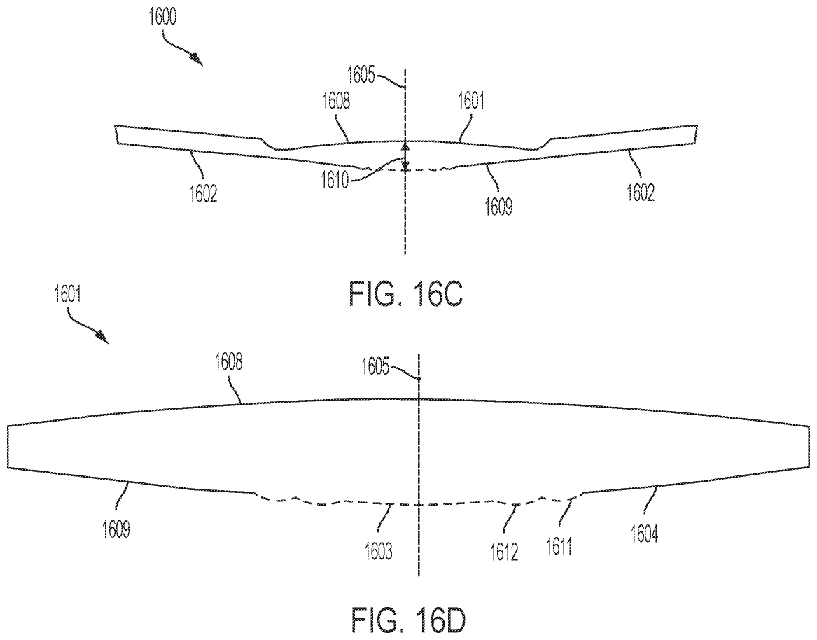

FIGS. 16A-16D illustrate a double-sided aspheric multifocal ophthalmic lens with phase-ring structures, according to some embodiments of the present disclosure. FIG. 16A shows a top view of a multifocal ophthalmic lens 1600, FIG. 16B shows a detailed view of the phase-ring structure of the multifocal ophthalmic lens 1600, FIG. 16C shows cross-sectional view of the multifocal ophthalmic lens 1600, and FIG. 16D shows a cross-sectional view of a lens body of the multifocal ophthalmic lens 1600.

Lens 1600 may include any one or more features of lens 1200 described above with respect to FIGS. 12A-12D. For example, lens 1600 may include a light transmissive circular disk-shaped lens body 1601 with an optic diameter 1606 and a center thickness 1610, as well as a pair of haptics 1602 as flexible support for the IOL when implanted into patient's eye, with a total outer diameter 1607. Lens body 1601 can include an anterior surface 1608, a posterior surface 1609, a central zone 1603 and a surrounding area 1604. As mentioned above, lens body 1601 can include an optical axis 1605 extending transverse to the anterior surface 1608 and posterior surface 1609. The pair of haptics 1602 can be extended outwardly from the lens body 1601 for supporting the lens 1600 after being implanted in the human eye. In some embodiments, the haptics 1602 of lens 1600 can hold the lens in place in the capsular bag.

In some embodiments, lens body 1601, like lens body 1201, can take the shape of a biconvex shape. Other shapes of lens body 1601 can include but are not limited to, plano-convex, biconcave, plano-concave shape, or combinations of convex and concave shapes. In some embodiments, both anterior surface 1608 and posterior surface 1609 can feature an aspheric structure, providing a double-sided asphericity for lens 1600.

As mentioned above at least with respect to FIGS. 13A-13B, lens 1600 can include a phase-ring structure comprising one or more regions. The phase-ring structure can be designed to provide a depth-of-focus via a non-diffracting beam principle with constructive phasing to maximize the focal energy. In some embodiments, the phase-ring structure of lens 1600 can include 1, 2, 3, 4, 5 or more regions. For example, lens 1600 illustrated at least in FIGS. 16A-16B may include an inner region (otherwise referred to herein as central zone 1603), an intermediate region 1612, and outer region 1611. Lens 1600 may include phase-ring structures on one of the surfaces or both surfaces of the lens. In some embodiments, the phase-ring structures can be placed on the posterior surface of the lens. As described above, each of the regions may comprise and/or be bounded by one or more rings extending concentrically with respect to an optical axis through the central zone 1603 (or inner region) over at least part of the posterior surface of the lens body 1601. In some embodiments, the regions may not be limited to concentric circular or annular ring-shaped regions but may instead or additionally include concentric elliptic or oval-shaped regions.

In some embodiments, the optic diameter 1606 of lens body 1601 may be greater than or equal to about 4 mm, such as about 4.5 mm, 5 mm, 5.5 mm, 6 mm, 6.5 mm, 7 mm, 7.5 mm, or 8 mm. The total outer diameter 1607 of lens 1600 including the haptics 1602 may be greater than or equal to about 9 mm, such as 10 mm, 11 mm, 12 mm, 13 mm, 14 mm, 15 mm, 16 mm, 17 mm, or 18 mm. Lens body 1601 may have a center thickness 1610 greater than or equal to about 0.8 mm, such as 0.85 mm, 0.9 mm, 0.95 mm, 1.0 mm, 1.05 mm, 1.10 mm, 1.15 mm, or 1.20 mm. One of ordinary skill in the art will recognize that although FIGS. 16A-16B illustrate an intraocular lens (IOL), other ophthalmic lenses, including multifocal diffractive contact lenses or eye glass lenses, could also benefit from the same approach. When used for ophthalmic multifocal contact lenses and spectacle or eye glass lenses, haptics 1602 may not be provided.

### Phase-Ring Structure Governing Equations

The phase-ring structures described above and embodied on the extended depth of focus ophthalmic lens and multifocal ophthalmic lens of the present disclosure can be designed using Equations (I) to (IV) as discussed below.

Pupil Function. A pupil function is a lens characteristic function that describes the physical effect of a lens by which it is possible to change the state of light made incident on the lens, and in specific terms, is represented by the product of the amplitude function A(r) and the exponential function of the phase function Φ₍ₙ₎(r) as noted in Equation (I) below.

$$T ( r ) = A ( r ) {e}^{i ( {Φ}_{( n )} ( r ) )} \qquad Equation ( I )$$

T(r): pupil function
A(r): amplitude function
Φ₍ₙ₎(r): phase function
n: natural number

Phase Function. A phase function is defined as the function that mathematically expresses the physical effect provided in a lens such as giving changes in the phase of incident light on a lens (position of wave peaks and valleys) using any method. The variable of the phase function is mainly expressed by position r in the radial direction from the center of the lens, and the phase of light made incident on the lens at the point of the position r undergoes a change by the phase function Φ₍ₙ₎(r) and is emitted from the lens. In specific terms, this is represented by an r−Φ coordinate system. In the present disclosure, phase is noted as Φ, and the unit is radians. One wavelength of light is represented as 2π radians, and a half wavelength as π radians, for example. A distribution of phase in the overall area in which the phase function is provided and expressed in the same coordinate system is called a phase profile, or simply a profile or zone profile. With an r axis of Φ=0 as a reference line, this means that the light made incident at the point of Φ=0 is emitted without changing the phase. Also, for this reference line, when a positive value is used for Φ, this means that progress of the light is delayed by that phase amount, and when a negative value is used for Φ, this means that progress of the light is advanced by that phase amount. In an actual ophthalmic lens, a refracting surface for which a diffractive structure is not given corresponds to this reference line (surface). Light undergoes a phase change based on this phase function and is emitted from the lens.

Amplitude Function. An amplitude function is the function expressed by A(r) in Equation (I) noted above. In the present disclosure, this is defined as a function that represents the change in the light transmission amount when passing through a lens. The variable of the amplitude function is represented as position r in the radial direction from the center of the lens, and represents the transmission rate of the lens at the point of position r. Also, the amplitude function is in a range of 0 or greater to 1 or less, which means that light is not transmitted at the point of A(r)=0, and that incident light is transmitted as it is without loss at the point of A(r)=1.

Zone. In the present disclosure, a zone is used as the minimum unit in a phase-ring structure, element, or diffraction grating provided in a lens. A zone may be circular or annular in shape and may be bounded along an inner radius and/or along an outer radius by a ring. For example, a first zone may be circular in area such that the ring is bounded only along an outer radius (i.e., a first ring), and for each subsequent zone the annular area between the (n−1)^{th} ring and the n^{th} ring may be referred to as the n^{th} zone.

The height profile of the phase-ring structure (Zₚₕₐₛₑ) on the ophthalmic lens can be calculated based on Equation (II) below. In some embodiments, the height may be based on one cycle (2π, which may be about 3.35 μm), the refractive index of the lens and the medium covering the lens, and the wavelength used (e.g., green light, which has a wavelength of about 550 nm).

$${Z}_{phase} ( r ) = {Φ}_{( n )} ( r ) \times \frac{λ}{{n}_{1} - {n}_{0}} \qquad Equation ( II )$$

Zₚₕₐₛₑ(r): height profile of the phase-ring structure
Φ₍ₙ₎(r): phase function
λ: design wavelength
n₁: refractive index of the lens material
n₀: refractive index of the medium covering the lens

The outer radius of a particular zone (rₙ) can be calculated based on Equation (III) below.

$${r}_{n} = \sqrt{2 \times λ \times n \times f} \qquad Equation ( III )$$

rₙ: outer radius of the n^{th} zone
λ: design wavelength
f: reciprocal of add power

Phase function (Φ₍ₙ₎(r)) can be calculated via Equation (IV) below.

$${Φ}_{( n )} ( r ) = {A}_{( n )} \times f ( \frac{r - {r}_{n}}{{r}_{n + 1} - {r}_{n}} ) + {D}_{( n )} \qquad Equation ( IV )$$

Φ₍ₙ₎(r): phase function
f(r): phase function base profile
r: radial distance from a center of lens
rₙ: outer radius of the n^{th} zone
rₙ₊₁: outer radius of the (n+1)^{th} zone

The phase function base profile f(r) could be a linear parabolic sine or polynomial function. A and D are the light distribution parameters. A is the amplitude scale factor; D is the vertical shift, if it is +D, the function moves up, if it is −D, then the function moves down. In some embodiments, A₍ₙ₎ can be a ratio of phase function base profile, and D₍ₙ₎ can be the phase shift of phase function base profile. As shown below with respect to the examples provided herein, the parameter A may correspond to the slope of the phase-ring structure in different zones.

The double-sided aspheric structure (anterior and posterior of the optic area of the ophthalmic lens) is for the correction of the spherical aberration of the lens. The height profile of the aspheric base structure (Zₐₛₚ) of the lens can be calculated according to the following Equation (V).

$${Z}_{asp} = \frac{{cr}^{2}}{1 + \sqrt{1 - ( 1 + k ) {c}^{2} {r}^{2}}} + {\sum }_{i = 2}^{n} {A}_{i} {r}^{2 i} \qquad Equation ( V )$$

Zₐₛₚ: height profile of the aspheric structure
r: radial distance from a center of lens
k: conic constant
c: curvature
Aᵢ: higher order aspheric coefficient

When both aspheric and phase-ring structures are placed onto the same surface (anterior surface and/or posterior surface of the ophthalmic lens), according to some embodiments of the present disclosure, the height profile of the combination structure (Zₜₒₜₐₗ) will be the summation of the height profile of the aspheric base curvature (Zₐₛₚ) and the height profile of the phase-ring structure (Zₚₕₐₛₑ), as calculated according to the below Equation (VI).

$${Z}_{total} ( r ) = {Z}_{asp} ( r ) + {Z}_{phase} ( r ) \qquad Equation ( VI )$$

Zₚₕₐₛₑ: height profile of the phase-ring structure
Zₐₛₚ: height profile of the aspheric structure
Zₜₒₜₐₗ: height profile of the combination structure, i.e., the lens body

In some embodiments, the above-described ophthalmic lenses can be a contact lens or an intraocular lens (IOL). In some embodiments, the IOL can be an intracorneal IOL, anterior chamber IOL, or posterior chamber IOL. While the haptic arms are illustrated in the embodiment, any suitable haptics fixation structure for the capsular bag or the ciliary sulcus compatible with posterior chamber implantation can also be used in a posterior chamber IOL.

A way of estimating the optical priority of an ophthalmic lens comprises determining experimentally its modulation transfer function (MTF). The MTF of an optical system can be measured according to Annex C of ISO 11979-2, which reflects the proportion of the contrast transmitted through the optical system for a determined spatial frequency of a test pattern, which frequency is defined as “cycles/mm” or “LP/mm”, in which “LP” indicates “line pairs.” Generally, the contrast decreases with an increase in spatial frequency.

Presented below are examples discussing different embodiments of the IOLs contemplated above. The following examples are provided to further illustrate the embodiments of the present disclosure but are not intended to limit the scope of the disclosure. While they are typical of those that might be used, other procedures, methodologies, or techniques known to those skilled in the art may alternatively be used.

### Examples

FIGS. 4-10 and 15A-15C illustrate the optical performance (MTF) of various ophthalmic lenses with phase-ring structures, in accordance at least with the below examples. As mentioned above, the phase-ring structures may comprise one or more zones, wherein zones may be bounded by one or more rings. For example, Equation (III) may be used to determine the radial distance from an optical axis to a given n^{th} ring on the lens.

The MTF of an extended depth of focus (EDOF) lens and multifocal lens may differ at varying apertures and/or resolution measurements. For example, at an aperture of 2 mm and resolution measurement of 50 LP/mm, an MTF value greater than 0.05 may indicate an EDOF lens design (e.g., curve (2) in FIGS. 4-10), evident by the single local maximum. On the other hand, an MTF value less than 0.05 may indicate a multifocal lens design (e.g., curve 2 in FIGS. 15A-C), evident by the two local maximums.

### Example 1: MTF of the EDOF Ophthalmic Lens According to a First Embodiment of the Present Disclosure

FIG. 4 illustrates an optical performance (MTF) of the extended depth of focus (EDOF) ophthalmic lens with phase-ring structures, according to a first embodiment of the present disclosure. The MTF of FIG. 4 may correspond with the phase profile illustrated in FIG. 2A. The parameters of A and D according to Equation (IV) (provided above) are varied according to Table 1 below.

TABLE 1
Variation of parameters A and D in a first
embodiment of the present disclosure.

| | A | D |
|---|---|---|
| Ring 1 | 0.1760 | −1.1352 |
| Ring 2 | 0.1056 | −0.9944 |
| Ring 3 | −0.2200 | −1.0516 |
| Ring 4 | 0.1056 | −1.1088 |
| Ring 5 | 0.2640 | −0.9240 |
| Ring 6 | 0.2640 | −0.6600 |
| Ring 7 | 0.2640 | −0.3960 |
| Ring 8 | 0.2640 | −0.1320 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. Curve (4) illustrates the MTF at a 2 mm aperture and at a resolution measurement of 50 LP/mm at a monofocal mode. The curves (1)-(4) in FIG. 4 show a focus at about 21.5 D with an add power

$$( \frac{1}{{f}_{1}} - \frac{1}{{f}_{0}} )$$

of about 2.4 D.

In some embodiments, each of the rings listed in Table 1 may correspond to a given point or set of points on a phase profile of a phase-ring structure. For example, FIG. 2A includes annotations (1)-(4) and (8), each of which may correspond respectively with rings 1˜4 and 8 in Table 1 above. As shown, ring 1 may correspond to the location at which the phase-ring structure transitions between a first inner sloping portion and a first outer sloping portion of the inner region. Ring 2 may correspond to the location at which the phase-ring structure transitions between the inner region and the outer region (i.e., the location at which the outer edge of the inner region is adjacent to the inner edge of the outer region). Ring 3 may correspond to the location at which the phase-ring structure transitions between a second inner sloping portion and an intermediate sloping portion of the outer region. Ring 4 may correspond to the location at which the phase-ring structure transitions from the intermediate sloping portion to a second outer sloping portion of the outer region. Ring 8 may correspond to the location at which the phase-ring structure of the lens transitions to the base curvature of the lens. Each of the rings not mentioned explicitly above may be located within a given sloping portion of the phase-ring structure. For example, ring 5, ring 6, and ring 7 may be located between ring 4 and ring 8 in the second outer sloping portion of the phase-ring structure.

### Example 2: MTF of the EDOF Ophthalmic Lens According to a Second Embodiment of the Present Disclosure

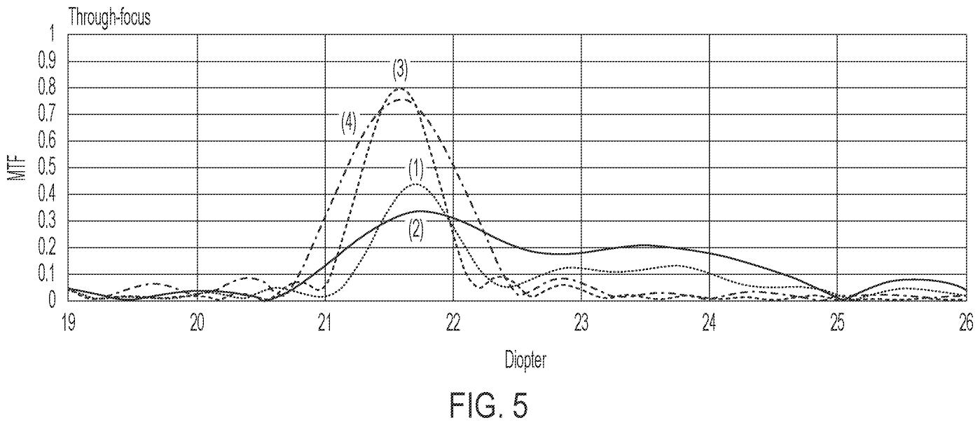

FIG. 5 illustrates the optical performance (MTF) of the EDOF ophthalmic lens with phase-ring structures, according to a second embodiment of the present disclosure. The MTF of FIG. 5 may correspond with the phase profile illustrated in FIG. 2A. The parameters of A and D according to Equation (IV) are varied according to Table 2 below.

TABLE 2
Variation of parameters A and D in a second
embodiment of the present disclosure.

| | A | D |
|---|---|---|
| Ring 1 | 0.134 | −1.0890 |
| Ring 2 | 0.124 | −0.9600 |
| Ring 3 | −0.262 | −1.0290 |
| Ring 4 | 0.06 | −1.1300 |
| Ring 5 | 0.3 | −0.9500 |
| Ring 6 | 0.3 | −0.6500 |
| Ring 7 | 0.26 | −0.3700 |
| Ring 8 | 0.24 | −0.1200 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. Curve (4) illustrates the MTF at a 2 mm aperture and at a resolution measurement of 50 LP/mm at a monofocal mode. The curves (1)-(4) in FIG. 5 show a focus at about 21.5 D with an add power

$$( \frac{1}{{f}_{1}} - \frac{1}{{f}_{0}} )$$

or about 2.4 D.

In some embodiments, each of the rings listed in Table 2 may correspond to a given point or set of points on a phase profile of a phase-ring structure. For example, FIG. 2A includes annotations (1)-(4) and (8), each of which may correspond respectively with rings 1˜4 and 8 in Table 2 above. The location of one or more of the rings from Table 2 in the phase profile of FIG. 2A may be substantially the same as that which is described above with respect to Example 1.

### Example 3: MTF of the EDOF Ophthalmic Lens According to a Third Embodiment of the Present Disclosure

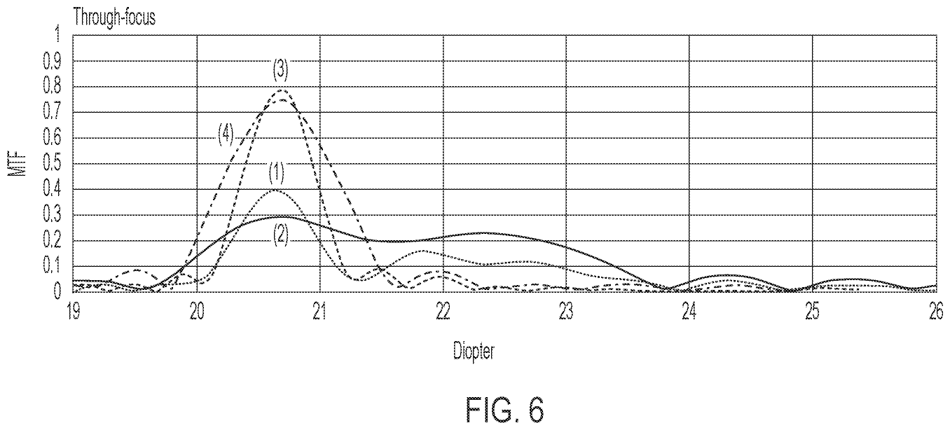

FIG. 6 illustrates the optical performance (MTF) of the EDOF ophthalmic lens with phase-ring structures, according to a third embodiment of the present disclosure. The MTF of FIG. 6 may correspond with the phase profile illustrated in FIG. 2A. The parameters of A and D according to Equation (IV) are varied according to Table 3 below.

TABLE 3
Variation of parameters A and D in a third
embodiment of the present disclosure.

| | A | D |
|---|---|---|
| Ring 1 | 0.1760 | −1.0166 |
| Ring 2 | 0.1056 | −0.8758 |
| Ring 3 | −0.2870 | −0.9665 |
| Ring 4 | 0.0700 | −1.0750 |
| Ring 5 | 0.2600 | −0.9100 |
| Ring 6 | 0.2600 | −0.6500 |
| Ring 7 | 0.2600 | −0.3900 |
| Ring 8 | 0.2600 | −0.1300 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. Curve (4) illustrates the MTF at a 2 mm aperture and at a resolution measurement of 50 LP/mm at a monofocal mode. The curves (1)-(4) in FIG. 6 show a focus at about 20.5 D with an add power

$$( \frac{1}{{f}_{1}} - \frac{1}{{f}_{0}} )$$

of about 2.4 D.

In some embodiments, each of the rings listed in Table 3 may correspond to a given point or set of points on a phase profile of a phase-ring structure. For example, FIG. 2A includes annotations (1)-(4) and (8), each of which may correspond respectively with rings 1˜4 and 8 in Table 3 above. The location of one or more of the rings from Table 3 in the phase profile of FIG. 2A may be substantially the same as that which is described above with respect to Examples 1 and/or 2.

### Example 4: MTF of the EDOF Ophthalmic Lens According to a Fourth Embodiment of the Present Disclosure

FIG. 7 illustrates the optical performance (MTF) of the EDOF ophthalmic lens with phase-ring structures, according to a fourth embodiment of the present disclosure. The MTF of FIG. 7 may correspond with the phase profile illustrated in FIG. 2C. The parameters of A and D according to Equation (IV) are varied according to Table 4 below.

TABLE 4
Variation of parameters A and D in a fourth
embodiment of the present disclosure.

| | A | D |
|---|---|---|
| Ring 1 | 0.2400 | −1.4300 |
| Ring 2 | 0.1600 | −1.2300 |
| Ring 3 | 0.1600 | −1.0700 |
| Ring 4 | −0.2000 | −1.0900 |
| Ring 5 | 0.0000 | −1.1900 |
| Ring 6 | 0.0000 | −1.1900 |
| Ring 7 | 0.0000 | −1.1900 |
| Ring 8 | 0.0000 | −1.1900 |
| Ring 9 | 0.2000 | −1.0900 |
| Ring 10 | 0.3300 | −0.8250 |
| Ring 11 | 0.3300 | −0.4950 |
| Ring 12 | 0.3300 | −0.1650 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. Curve (4) illustrates the MTF at a 2 mm aperture and at a resolution measurement of 50 LP/mm at a monofocal mode. The curves (1)-(4) in FIG. 7 show a focus at about 20.5 D with an add power

$$( \frac{1}{{f}_{1}} - \frac{1}{{f}_{0}} )$$

of about 3.35 D.

In some embodiments, each of the rings listed in Table 4 may correspond to a given point or set of points on a phase profile of a phase-ring structure. For example, FIG. 2C includes annotations (1), (3), (4), (8), (9), and (12), each of which may correspond respectively with rings 1, 3, 4, 8, 9, and 12 in Table 4 above. As shown, ring 1 may correspond to the location at which the phase-ring structure transitions between a first inner sloping portion and a first outer sloping portion of the inner region. Ring 3 may correspond to the location at which the phase-ring structure transitions between the inner region and the outer region (i.e., the location at which the outer edge of the inner region is adjacent to the inner edge of the outer region). Ring 4 may correspond to the location at which the phase-ring structure transitions between a second inner sloping portion and a first intermediate sloping portion. Ring 8 may correspond to the location at which the phase-ring structure transitions from the first intermediate sloping portion to a second intermediate sloping portion of the outer region. Ring 9 may correspond to the location at which the phase-ring structure transitions from the second intermediate sloping portion to a second outer sloping portion of the outer region. Ring 12 may correspond to the location at which the phase-ring structure of the lens transitions to the base curvature of the lens. Each of the rings not mentioned explicitly above may be located within a given sloping portion of the phase-ring structure. For example, ring 2 may be located between ring 1 and 3 in the first outer sloping portion of the phase-ring structure. Ring 5, ring 6, and ring 7 may be located between ring 4 and ring 8 in the first intermediate sloping portion of the phase-ring structure. Ring 10 and ring 11 may be located between ring 9 and ring 12 in the second outer sloping portion of the phase-ring structure.

### Example 5: MTF of the EDOF Ophthalmic Lens According to a Fifth Embodiment of the Present Disclosure

FIG. 8 illustrates the optical performance (MTF) of the EDOF ophthalmic lens with phase-ring structures, according to a fifth embodiment of the present disclosure. The MTF of FIG. 8 may correspond with the phase profile illustrated in FIG. 2C. The parameters of A and D according to Equation (IV) are varied according to Table 5 below.

TABLE 5
Variation of parameters A and D in a fifth
embodiment of the present disclosure.

| | A | D |
|---|---|---|
| Ring 1 | 0.2400 | −1.4300 |
| Ring 2 | 0.1600 | −1.2300 |
| Ring 3 | 0.1600 | −1.0700 |
| Ring 4 | −0.3200 | −1.1500 |
| Ring 5 | 0.0300 | −1.2950 |
| Ring 6 | 0.0300 | −1.2650 |
| Ring 7 | 0.0300 | −1.2350 |
| Ring 8 | 0.0300 | −1.2050 |
| Ring 9 | 0.2000 | −1.0900 |
| Ring 10 | 0.3300 | −0.8250 |
| Ring 11 | 0.3300 | −0.4950 |
| Ring 12 | 0.3300 | −0.1650 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. Curve (4) illustrates the MTF at a 2 mm aperture and at a resolution measurement of 50 LP/mm at a monofocal mode. The curves (1)-(4) in FIG. 8 show a focus at about 20.5 D with an add power

$$( \frac{1}{{f}_{1}} - \frac{1}{{f}_{0}} )$$

of about 3.35 D.

In some embodiments, each of the rings listed in Table 5 may correspond to a given point or set of points on a phase profile of a phase-ring structure. For example, FIG. 2C includes annotations (1), (3), (4), (8), (9), and (12), each of which may correspond respectively with rings 1, 3, 4, 8, 9, and 12 in Table 5 above. The location of one or more of the rings from Table 5 in the phase profile of FIG. 2C may be substantially the same as that which is described above with respect to Example 4.

### Example 6: MTF of the EDOF Ophthalmic Lens According to a Sixth Embodiment of the Present Disclosure

FIG. 9 illustrates the optical performance (MTF) of the EDOF ophthalmic lens with phase-ring structures, according to a sixth embodiment of the present disclosure. The MTF of FIG. 9 may correspond with the phase profile illustrated in FIG. 2B. The parameters of A and D according to Equation (IV) are varied according to Table 6 below.

TABLE 6
Variation of parameters A and D in a sixth
embodiment of the present disclosure.

| | A | D |
|---|---|---|
| Ring 1 | 0.2200 | −1.4100 |
| Ring 2 | 0.2000 | −1.2000 |
| Ring 3 | 0.2000 | −1.0000 |
| Ring 4 | −0.3200 | −1.0600 |
| Ring 5 | 0.0300 | −1.2050 |
| Ring 6 | 0.0300 | −1.1750 |
| Ring 7 | 0.0300 | −1.1450 |
| Ring 8 | 0.0300 | −1.1150 |
| Ring 9 | 0.2000 | −1.0000 |
| Ring 10 | 0.3000 | −0.7500 |
| Ring 11 | 0.3000 | −0.4500 |
| Ring 12 | 0.3000 | −0.1500 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. Curve (4) illustrates the MTF at a 2 mm aperture and at a resolution measurement of 50 LP/mm at a monofocal mode. The curves (1)-(4) in FIG. 9 show a focus at about 20.5 D with an add power

$$( \frac{1}{{f}_{1}} - \frac{1}{{f}_{0}} )$$

of about 3.0 D.

In some embodiments, each of the rings listed in Table 6 may correspond to a given point or set of points on a phase profile of a phase-ring structure. For example, FIG. 2B includes annotations (1), (3), (4), (8), (9), and (12), each of which may correspond respectively with rings 1, 3, 4, 8, 9, and 12 in Table 6 above. As shown, ring 1 may correspond to the location at which the phase-ring structure transitions between a first inner sloping portion and a first outer sloping portion of the inner region. Ring 3 may correspond to the location at which the phase-ring structure transitions between the inner region and the outer region (i.e., the location at which the outer edge of the inner region is adjacent to the inner edge of the outer region). Ring 4 may correspond to the location at which the phase-ring structure transitions between a second inner sloping portion and a first intermediate sloping portion. Ring 8 may correspond to the location at which the phase-ring structure transitions from the first intermediate sloping portion to a second intermediate sloping portion of the outer region. Ring 9 may correspond to the location at which the phase-ring structure transitions from the second intermediate sloping portion to a second outer sloping portion of the outer region. Ring 12 may correspond to the location at which the phase-ring structure of the lens transitions to the base curvature of the lens. Each of the rings not mentioned explicitly above may be located within a given sloping portion of the phase-ring structure. For example, ring 2 may be located between ring 1 and 3 in the first outer sloping portion of the phase-ring structure. Ring 5, ring 6, and ring 7 may be located between ring 4 and ring 8 in the first intermediate sloping portion of the phase-ring structure. Ring 10 and ring 11 may be located between ring 9 and ring 12 in the second outer sloping portion of the phase-ring structure.

### Example 7: MTF of the EDOF Ophthalmic Lens According to a Seventh Embodiment of the Present Disclosure

FIG. 10 illustrates the optical performance (MTF) of the EDOF ophthalmic lens with phase-ring structures, according to a seventh embodiment of the present disclosure. The MTF of FIG. 10 may correspond with the phase profile illustrated in FIG. 2B. The parameters of A and D according to Equation (IV) are varied according to Table 7 below.

TABLE 7
Variation of parameters A and D in a seventh
embodiment of the present disclosure.

| | A | D |
|---|---|---|
| Ring 1 | 0.2400 | −1.4600 |
| Ring 2 | 0.2200 | −1.2300 |
| Ring 3 | 0.2200 | −1.0100 |
| Ring 4 | −0.3800 | −1.0900 |
| Ring 5 | 0.0300 | −1.2650 |
| Ring 6 | 0.0300 | −1.2350 |
| Ring 7 | 0.0300 | −1.2050 |
| Ring 8 | 0.0300 | −1.1750 |
| Ring 9 | 0.2600 | −1.0300 |
| Ring 10 | 0.3000 | −0.7500 |
| Ring 11 | 0.3000 | −0.4500 |
| Ring 12 | 0.3000 | −0.1500 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. Curve (4) illustrates the MTF at a 2 mm aperture and at a resolution measurement of 50 LP/mm at a monofocal mode. The curves (1)-(4) in FIG. 10 show a focus at about 20.5 D with an add power

$$( \frac{1}{{f}_{1}} - \frac{1}{{f}_{0}} )$$

of about 3.0 D.

In some embodiments, each of the rings listed in Table 7 may correspond to a given point or set of points on a phase profile of a phase-ring structure. For example, FIG. 2B includes annotations (1), (3), (4), (8), (9), and (12), each of which may correspond respectively with rings 1, 3, 4, 8, 9, and 12 in Table 7 above. The location of one or more of the rings from Table 7 in the phase profile of FIG. 2B may be substantially the same as that which is described above with respect to Example 6.

### Example 8: MTF of the Multifocal Lens According to a First Embodiment of the Present Disclosure

FIG. 15A illustrates the optical performance (MTF) of the multifocal ophthalmic lens with phase-ring structures, according to a first embodiment of the present disclosure. The MTF of FIG. 15A may correspond with the phase profile illustrated in FIG. 13A. The parameters of A and D according to Equation (IV) (provided above) are varied according to Table 8 below.

TABLE 8
Variation of parameters A and D in a first
embodiment of the present disclosure.

| | A | D |
|---|---|---|
| Ring 1 | 0.2400 | −1.2000 |
| Ring 2 | 0.2400 | −0.9600 |
| Ring 3 | −0.4800 | −1.0800 |
| Ring 4 | 0.1300 | −1.2550 |
| Ring 5 | 0.1300 | −1.1250 |
| Ring 6 | 0.1300 | −0.9950 |
| Ring 7 | −0.3900 | −1.1250 |
| Ring 8 | 0.1600 | −1.2400 |
| Ring 9 | 0.1600 | −1.0800 |
| Ring 10 | 0.3400 | −0.8300 |
| Ring 11 | 0.3300 | −0.4950 |
| Ring 12 | 0.3300 | −0.1650 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. The curves (1)-(3) in FIG. 15A show a focus at about 21.5 D with an add power

$$( \frac{1}{{f}_{1}} - \frac{1}{{f}_{0}} )$$

of about 3.5 D.

In some embodiments, each of the rings listed in Table 8 may correspond to a given point or set of points on a phase profile of a phase-ring structure. For example, FIG. 13A includes annotations (1)-(3), (6), (7), (9), and (12), each of which may correspond respectively with rings 1-3, 6, 7, 9, and 12 in Table 8 above. As shown, ring 1 may correspond to the location at which the phase-ring structure transitions between a first inner sloping portion and a first outer sloping portion of the inner region. Ring 2 may correspond to the location at which the phase-ring structure transitions between the inner region and an intermediate region (i.e., the location at which the outer edge of the inner region is adjacent to the inner edge of the intermediate region). Ring 3 may correspond to the location at which the phase-ring structure transitions between a second inner sloping portion and a second outer sloping portion of the intermediate region. Ring 6 may correspond to the location at which the phase-ring structure transitions between the intermediate region and the outer region (i.e., the location at which the outer edge of the intermediate region is adjacent to the inner edge of the outer region). Ring 7 may correspond to the location at which the phase-ring structure transitions from a third inner sloping portion to an intermediate sloping portion of the outer region. Ring 9 may correspond to the location at which the phase-ring structure transitions from the intermediate sloping portion to a third outer sloping portion of the outer region. Ring 12 may correspond to the location at which the phase-ring structure of the lens transitions to the base curvature of the lens. Each of the rings not mentioned explicitly above may be located within a given sloping portion of the phase-ring structure. For example, rings 4 and 5 may be located between ring 3 and 6 in the second outer sloping portion of the phase-ring structure. Ring 8 may be located between ring 7 and ring 9 in the intermediate sloping portion of the phase-ring structure. Ring 10 and ring 11 may be located between ring 9 and ring 12 in the third outer sloping portion of the phase-ring structure.

### Example 9: MTF of the Multifocal Lens According to a Second Embodiment of the Present Disclosure

FIG. 15B illustrates the optical performance (MTF) of the multifocal ophthalmic lens with phase-ring structures, according to a second embodiment of the present disclosure. The MTF of FIG. 15B may correspond with the phase profile illustrated in FIG. 13A. The parameters of A and D according to Equation (IV) (provided above) are varied according to Table 9 below.

TABLE 9
Variation of parameters A and D in a second
embodiment of the present disclosure.

| | A | D |
|---|---|---|
| Ring 1 | 0.2400 | −1.3490 |
| Ring 2 | 0.2400 | −1.1090 |
| Ring 3 | −0.4000 | −1.1890 |
| Ring 4 | 0.1300 | −1.3240 |
| Ring 5 | 0.1300 | −1.1940 |
| Ring 6 | 0.1300 | −1.0640 |
| Ring 7 | −0.4000 | −1.1990 |
| Ring 8 | 0.2000 | −1.2990 |
| Ring 9 | 0.2000 | −1.0990 |
| Ring 10 | 0.3330 | −0.8325 |
| Ring 11 | 0.3330 | −0.4995 |
| Ring 12 | 0.3330 | −0.1665 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. The curves (1)-(3) in FIG. 15B show a focus at about 21.5 D with an add power

$$( \frac{1}{{f}_{1}} - \frac{1}{{f}_{0}} )$$

of about 3.5 D.

In some embodiments, each of the rings listed in Table 9 may correspond to a given point or set of points on a phase profile of a phase-ring structure. For example, FIG. 13A includes annotations (1)-(3), (6), (7), (9), and (12), each of which may correspond respectively with rings 1-3, 6, 7, 9, and 12 in Table 9 above. The location of one or more of the rings from Table 9 in the phase profile of FIG. 13A may be substantially the same as that which is described above with respect to Example 8.

### Example 10: MTF of the Multifocal Lens According to a Fifth Embodiment of the Present Disclosure

FIG. 15C illustrates the optical performance (MTF) of the multifocal ophthalmic lens with phase-ring structures, according to a fifth embodiment of the present disclosure. The MTF of FIG. 15C may correspond with the phase profile illustrated in FIG. 13B. The parameters of A and D according to Equation (IV) (provided above) are varied according to Table 10 below.

TABLE 10
Variation of parameters A and D in a fifth
embodiment of the present disclosure.

| | A | D |
|---|---|---|
| Ring 1 | 0.2000 | −1.2000 |
| Ring 2 | 0.1200 | −1.0400 |
| Ring 3 | 0.1000 | −0.9300 |
| Ring 4 | −0.5400 | −1.1500 |
| Ring 5 | 0.2200 | −1.3100 |
| Ring 6 | 0.2200 | −1.0900 |
| Ring 7 | −0.2600 | −1.1100 |
| Ring 8 | 0.0600 | −1.2100 |
| Ring 9 | 0.0600 | −1.1500 |
| Ring 10 | 0.0400 | −1.1000 |
| Ring 11 | 0.3600 | −0.9000 |
| Ring 12 | 0.3600 | −0.5400 |
| Ring 13 | 0.3600 | −0.1800 |

Curve (1) illustrates the MTF at a 3 mm aperture and at a resolution measurement of 50 LP/mm. Curve (2) illustrates the MTF at a 2 mm aperture and at a resolution measurement of 50 LP/mm. Curve (3) illustrates the MTF at a 3 mm aperture and at a resolution measurement of 50 LP/mm at a monofocal mode. The curves (1)-(3) in FIG. 15C show a focus at about 20.5 D with an add power

$$( \frac{1}{{f}_{1}} - \frac{1}{{f}_{0}} )$$

of about 3.35 D.

In some embodiments, each of the rings listed in Table 10 may correspond to a given point or set of points on a phase profile of a phase-ring structure. For example, FIG. 13B includes annotations (1), (3), (4), (6), (7), (10), and (13), each of which may correspond respectively with rings 1, 3, 4, 6, 7, 10, and 13 in Table 10 above. As shown, ring 1 may correspond to the location at which the phase-ring structure transitions between a first inner sloping portion and a first outer sloping portion of the inner region. Ring 3 may correspond to the location at which the phase-ring structure transitions between the inner region and an intermediate region (i.e., the location at which the outer edge of the inner region is adjacent to the inner edge of the intermediate region). Ring 4 may correspond to the location at which the phase-ring structure transitions between a second inner sloping portion and a second outer sloping portion of the intermediate region. Ring 6 may correspond to the location at which the phase-ring structure transitions between the intermediate region and the outer region (i.e., the location at which the outer edge of the intermediate region is adjacent to the inner edge of the outer region). Ring 7 may correspond to the location at which the phase-ring structure transitions from a third inner sloping portion to an intermediate sloping portion of the outer region. Ring 10 may correspond to the location at which the phase-ring structure transitions from the intermediate sloping portion to a third outer sloping portion of the outer region. Ring 13 may correspond to the location at which the phase-ring structure of the lens transitions to the base curvature of the lens. Each of the rings not mentioned explicitly above may be located within a given sloping portion of the phase-ring structure. For example, ring 2 may be located between ring 1 and 3 in the first outer sloping portion of the phase-ring structure. Ring 5 may be located between ring 4 and ring 6 in the second outer sloping portion of the phase-ring structure. Ring 8 and ring 9 may be located between ring 7 and ring 10 in the intermediate sloping portion of the phase-ring structure. Ring 11 and ring 12 may be located in the third outer sloping portion of the phase-ring structure.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The numerical ranges disclosed inherently support any range or value within the disclosed numerical ranges, including the endpoints, even though a precise range limitation is not stated verbatim in the specification because this disclosure can be practiced throughout the disclosed numerical ranges.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EMBODIMENTS

Embodiment 1. An ophthalmic lens comprising a lens body, the lens body comprising:
   (a) a first aspheric surface;
   (b) a second aspheric surface (Zₜₒₜₐₗ) including a base curve (Zₐₛₚ) and a phase-ring structure (Zₚₕₐₛₑ) composed of phase profile Φ₍ₙ₎(r), thereby producing a distance focus (f₀) and an extended focus (f₁).
Embodiment 2. The ophthalmic lens of embodiment 1, wherein the first aspheric surface is anterior surface.
Embodiment 3. The ophthalmic lens of embodiment 1, wherein the second aspheric surface is posterior surface.
Embodiment 4. The ophthalmic lens of embodiment 1, wherein the first aspheric surface comprises a toric component.
Embodiment 5. The ophthalmic lens of embodiment 1, wherein a height profile of the base curve (Zₐₛₚ) is represented by:

$${Z}_{asp} = \frac{{cr}^{2}}{1 + \sqrt{1 - ( 1 + k ) {c}^{2} {r}^{2}}} + {\sum }_{i = 2}^{n} {A}_{i} {r}^{2 i}$$

wherein Zₐₛₚ is the height profile of the aspheric structure, r is the radial distance from the center of the lens in millimeters, c is the curvature, k is the conic constant, and Aᵢ are higher order correction terms.
Embodiment 6. The ophthalmic lens of embodiment 1, wherein the second aspheric surface (Zₜₒₜₐₗ) is represented by:

$${Z}_{total} ( r ) = {Z}_{asp} ( r ) + {Z}_{phase} ( r ) .$$

Embodiment 7. The ophthalmic lens of embodiment 1, wherein the phase profile Φ₍ₙ₎(r) is represented by:

$${Φ}_{( n )} ( r ) = {A}_{( n )} \times f ( \frac{r - {r}_{n}}{{r}_{n + 1} - {r}_{n}} ) + {D}_{( n )}$$

where r is the radial distance of the lens in millimeter, rₙ is radius of n^{th} zone, rₙ₊₁ is radius of (n+1)^{th} zone, f(r) is the phase function base profile being a linear, parabolic, sine or polynomial function, A₍ₙ₎ is a ratio of phase function base profile, and D₍ₙ₎ is the phase shift of phase function base profile.
Embodiment 8. The ophthalmic lens of embodiment 7, wherein the radius of the n^{th} zone (rₙ) is represented by:

$${r}_{n} = \sqrt{2 \times λ \times n \times f}$$

rₙ is the radius of the n^{th} zone, λ is the design wavelength, and f is the reciprocal of add power.
Embodiment 9. The ophthalmic lens of embodiment 1, wherein the height profile of the phase-ring structure is relative to the base curvature of the ophthalmic lens:

$${Z}_{phase} ( r ) = {Φ}_{( n )} ( r ) \times \frac{λ}{{n}_{1} - {n}_{o}}$$

wherein Zₚₕₐₛₑ is the height profile of the phase-ring structure, λ is the design wavelength, Φ₍ₙ₎(r) is phase profile, n₁ is refractive index of the ophthalmic lens material, and n₀ is refractive index of a medium covering the ophthalmic lens.
Embodiment 10. The ophthalmic lens of embodiment 1, wherein a phase profile Φ₍ₙ₎(r) is in the range of:

$$0 \leq \vert {Φ}_{( n )} ( r ) \vert \leq 6 π .$$

Embodiment 11. The ophthalmic lens of embodiment 1, wherein the distance focus (f₀) and the extended focus (f₁) are in the range of:

$$- 15 D \leq \frac{1}{{f}_{0}} \leq 55 D , \frac{1}{6 f} \leq \frac{1}{{f}_{1}} - \frac{1}{{f}_{0}} \leq \frac{1}{2 f} , and 3 D \leq \frac{1}{f} \leq 12 D .$$

Embodiment 12. The ophthalmic lens of embodiment 1, wherein the ophthalmic lens is an intraocular lens (IOL) or contact lens.
Embodiment 13. The ophthalmic lens of embodiment 1, further comprising a pair of haptics extended outwardly from the lens body.
Embodiment 14a. A method of treating an ophthalmic disease or disorder in a subject, the method comprising implanting into an eye of the subject an ophthalmic lens comprising a lens body, the lens body comprising:
   (a) a base curve; and
   (b) a phase-ring structure.
Embodiment 14b. A method of treating an ophthalmic disease or disorder in a subject, the method comprising implanting into an eye of the subject an ophthalmic lens comprising a lens body, the lens body comprising:
   (a) a first aspheric surface; and
   (b) a second aspheric surface comprising a base curve and an optical phase-ring structure.
Embodiment 15. The method of embodiment 14, wherein the ophthalmic disease or disorder is selected from the group consisting of cataract and presbyopia.
Embodiment 16. The method of embodiment 14, wherein the ophthalmic lens is an IOL or contact lens.
Embodiment 17. The method of embodiment 16, wherein the IOL further comprises a pair of haptics extended outwardly from the lens body.
Embodiment 18. The method of embodiment 16, wherein the IOL is implanted into a capsular bag of the subject's eye.
Embodiment 19. A method of manufacturing an ophthalmic lens, the method comprising:
   (a) manufacturing a first aspheric surface optionally comprising a toric component; and
   (b) manufacturing a second aspheric surface comprising a base curve and a phase-ring structure,
   thereby producing a distance focus (f₀) and an extended focus (f₁).
Embodiment 20. The method of embodiment 19, further comprising:
   performing an in-situ image quality analysis to ensure the performance of the ophthalmic lens meets the pre-established quality criteria.
Embodiment 21. An ophthalmic lens comprising a lens body, the lens body comprising:
   a first aspheric surface; and
   a second aspheric surface comprising a base curvature and a phase-ring structure, wherein the phase-ring structure comprises an inner region and an outer region, and
      the inner region comprises a first phase-ring surface having a first curvature;
      the outer region comprises a second phase-ring surface having a second curvature; and
      an outer edge of the inner region is adjacent to an inner edge of the outer region,
   wherein refraction of light passing through the inner region and the outer region causes constructive interference that produces a distance focus (f₀) and an extended focus (f₁).
Embodiment 22. The ophthalmic lens of embodiment 21, wherein a radius of the inner region of the phase-ring structure that extends from an optical axis of the ophthalmic lens to the outer edge of the inner region is greater than 0 mm and less than or equal to 0.70 mm.
Embodiment 23. The ophthalmic lens of embodiment 21, wherein a radius of the outer region of the phase-ring structure that extends from an optical axis of the ophthalmic lens to an outer edge of the outer region is greater than 0 mm and less than or equal to 1.30 mm.
Embodiment 24. The ophthalmic lens of embodiment 21, wherein the first curvature of the first phase-ring surface comprises a first inner sloping portion that slopes at a first angle with respect to an optical axis of the ophthalmic lens and a first outer sloping portion that slopes at a second angle with respect to the optical axis.
Embodiment 25. The ophthalmic lens of embodiment 24, wherein the first inner sloping portion and the first outer sloping portion together slope monotonically with respect to the optical axis.
Embodiment 26. The ophthalmic lens of embodiment 21, wherein the second curvature of the second phase-ring surface comprises a second inner sloping portion that slopes at a third angle with respect to an optical axis of the ophthalmic lens, an intermediate sloping portion that slopes at a fourth angle with respect to the optical axis, and a second outer sloping portion that slopes at a fifth angle with respect to the optical axis.
Embodiment 27. The ophthalmic lens of embodiment 26, wherein the intermediate sloping portion and the second outer sloping portion together slope monotonically with respect to the optical axis, and the intermediate portion and the second inner sloping portion together do not slope monotonically with respect to the second outer sloping portion.
Embodiment 28. The ophthalmic lens of embodiment 21, wherein the first aspheric surface is an anterior surface of the lens body.
Embodiment 29. The ophthalmic lens of embodiment 21, wherein the second aspheric surface is a posterior surface of the lens body.
Embodiment 30. The ophthalmic lens of embodiment 21, wherein the first aspheric surface comprises a toric component.
Embodiment 31. The ophthalmic lens of embodiment 21, wherein a height profile (Zₐₛₚ) of the base curvature is represented by:

$${Z}_{asp} = \frac{{cr}^{2}}{1 + \sqrt{1 - ( 1 + k ) {c}^{2} {r}^{2}}} + {\sum }_{i = 2}^{n} {A}_{i} {r}^{2 i}$$

wherein r is a radial distance from the center of the lens in millimeters, c is a lens curvature, k is a conic constant, and Aᵢ is a higher order correction term.
Embodiment 32. The ophthalmic lens of embodiment 21, wherein a height profile of the lens body (Zₜₒₜₐₗ) is represented by:

$${Z}_{total} ( r ) = {Z}_{asp} ( r ) + {Z}_{phase} ( r ) .$$

Embodiment 33. The ophthalmic lens of embodiment 21, wherein the phase profile (Φ₍ₙ₎(r)) is represented by:

$${Φ}_{( n )} ( r ) = {A}_{( n )} \times f ( \frac{r - {r}_{n}}{{r}_{n + 1} - {r}_{n}} ) + {D}_{( n )} ,$$

wherein r is the radial distance from the center of the lens in millimeters, rₙ is an outer radius of an n^{th} zone, rₙ₊₁ is an outer radius of an (n+1)^{th} zone, f(r) is a phase function base profile, the phase function base profile being a linear, parabolic, sine or polynomial function, A₍ₙ₎ is a ratio of the phase function base profile, and D₍ₙ₎ is a phase shift of the phase function base profile.
Embodiment 34. The ophthalmic lens of embodiment 33, wherein the outer radius of the n^{th} zone (rₙ) is represented by:

$${r}_{n} = \sqrt{2 \times λ \times n \times f} ,$$

wherein λ is a design wavelength, and f is a reciprocal of add power.
Embodiment 35. The ophthalmic lens of embodiment 33, wherein a height ratio between the inner region and the outer region of the phase-ring structure is represented by:

$$0 \leq {Φ}_{( n )} ( {r}_{out} ) - {Φ}_{( n )} ( {r}_{in} ) \leq 4 π .$$

Embodiment 36. The ophthalmic lens of embodiment 21, wherein a height profile (Zₚₕₐₛₑ) of the phase-ring structure is relative to the base curvature of the ophthalmic lens:

$${Z}_{phase} ( r ) = {Φ}_{( n )} ( r ) \times \frac{λ}{{n}_{1} - {n}_{0}}$$

wherein λ is a design wavelength, Φ₍ₙ₎(r) is a phase profile, n₁ is a refractive index of a material of the ophthalmic lens, and no is a refractive index of a medium covering the ophthalmic lens.
Embodiment 37. The ophthalmic lens of embodiment 21, wherein a phase profile Φ₍ₙ₎(r)) of the phase-ring structure is between:

$$0 \leq \vert {Φ}_{( n )} ( r ) \vert \leq 8 π$$

Embodiment 38. The ophthalmic lens of embodiment 21, wherein the distance focus (f₀) and the extended focus (f₁) are defined by the following expressions:

$$- 15 D \leq \frac{1}{{f}_{0}} \leq 55 D ,$$

$$\frac{1}{6 f} \leq \frac{1}{{f}_{1}} - \frac{1}{{f}_{0}} \leq \frac{1}{2 f} ,$$

$$and$$

$$3 D \leq \frac{1}{f} \leq 16 D ,$$

wherein

$$\frac{1}{f}$$

is add power.
Embodiment 39. The ophthalmic lens of embodiment 21, wherein the ophthalmic lens is an intraocular lens or a contact lens.
Embodiment 40. The ophthalmic lens of embodiment 21, comprising a pair of haptics extended outwardly from the lens body.
Embodiment 41. A method of treating an ophthalmic disease or disorder in a subject, the method comprising implanting into an eye of the subject an ophthalmic lens comprising a lens body, the lens body comprising:
   a first aspheric surface; and
   a second aspheric surface comprising a base curvature and a phase-ring structure, wherein the phase-ring structure comprises an inner region and an outer region, and
      the inner region comprises a first phase-ring surface having a first curvature;
      the outer region comprises a second phase-ring surface having a second curvature; and
      an outer edge of the inner region is adjacent to an inner edge of the outer region,
   wherein refraction of light passing through the inner region and the outer region causes constructive interference that produces a distance focus and an extended focus.
Embodiment 42. The method of embodiment 41, wherein the ophthalmic disease or disorder is selected from the group consisting of: cataract and presbyopia.
Embodiment 43. The method of embodiment 41, wherein the ophthalmic lens is an intraocular lens or a contact lens.
Embodiment 44. The method of embodiment 43, wherein the intraocular lens comprises a pair of haptics extended outwardly from the lens body.
Embodiment 45. The method of embodiment 43, wherein the intraocular lens is implanted into a capsular bag of the eye of the subject.
Embodiment 46. A method of manufacturing an ophthalmic lens, the method comprising:
   manufacturing a first aspheric surface;
   manufacturing a second aspheric surface comprising a base curvature; and
   generating a phase-ring structure on the second aspheric surface, wherein the phase-ring structure comprises an inner region and an outer region, and
      the inner region comprises a first phase-ring surface having a first curvature;
      the outer region comprises a second phase-ring surface having a second curvature; and
      an outer edge of the inner region is adjacent to an inner edge of the outer region,
   wherein refraction of light passing through the inner region and the outer region causes constructive interference that produces a distance focus and an extended focus.
Embodiment 47. The method of embodiment 46, comprising performing an in-situ image quality analysis to determine whether a performance of the ophthalmic lens meets predetermined quality criteria.
Embodiment 48. An ophthalmic lens comprising a lens body, the lens body comprising:
   a first aspheric surface; and
   a second aspheric surface comprising a base curvature and a phase-ring structure, wherein the phase-ring structure comprises an inner region, an intermediate region, and an outer region, and
      the inner region comprises a first phase-ring surface having a first curvature;
      the intermediate region comprises a second phase-ring surface having a second curvature;
      the outer region comprises a third phase-ring surface having a third curvature;
      an outer edge of the inner region is adjacent to an inner edge of the intermediate region; and
      an outer edge of the intermediate region is adjacent to an inner edge of the outer region,
   wherein refraction of light passing through the inner region, the intermediate region, and the outer region causes constructive interference that produces a near focus and a distance focus.
Embodiment 49. The ophthalmic lens of embodiment 48, wherein a radius of the inner region of the phase-ring structure that extends from an optical axis of the ophthalmic lens to the outer edge of the inner region is greater than 0 mm and less than or equal to 0.70 mm.
Embodiment 50. The ophthalmic lens of embodiment 48, wherein a radius of the intermediate region of the phase-ring structure that extends from an optical axis of the ophthalmic lens to the outer edge of the intermediate region is greater than 0 mm and less than or equal to 1.30 mm.
Embodiment 51. The ophthalmic lens of embodiment 48, wherein a radius of the outer region of the phase-ring structure that extends from an optical axis of the ophthalmic lens to an outer edge of the outer region is greater than 0 mm and less than or equal to 1.30 mm.
Embodiment 52. The ophthalmic lens of embodiment 48, wherein the first curvature of the first phase-ring surface comprises a first inner sloping portion that slopes at a first angle with respect to an optical axis of the ophthalmic lens and a first outer sloping portion that slopes at a second angle with respect to the optical axis.
Embodiment 53. The ophthalmic lens of embodiment 52, wherein the first inner sloping portion and the first outer sloping together portion slope monotonically with respect to the optical axis.
Embodiment 54. The ophthalmic lens of embodiment 48, wherein the second curvature of the second phase-ring surface comprises a second inner sloping portion that slopes at a third angle with respect to an optical axis of the ophthalmic lens and a second outer sloping portion that slopes at a fourth angle with respect to the optical axis.
Embodiment 55. The ophthalmic lens of embodiment 54, wherein the second inner sloping portion and the second outer sloping portion together do not slope monotonically with respect to the optical axis.
Embodiment 56. The ophthalmic lens of embodiment 48, wherein the third curvature of the third phase-ring surface comprises a third inner sloping portion that slopes at a fifth angle with respect to an optical axis of the ophthalmic lens and a third outer sloping portion that slopes at a sixth angle with respect to the optical axis.
Embodiment 57. The ophthalmic lens of embodiment 56, wherein the third inner sloping portion and the third outer sloping portion together do not slope monotonically with respect to the optical axis.
Embodiment 58. The ophthalmic lens of embodiment 57, wherein the first aspheric surface is an anterior surface of the lens body.
Embodiment 59. The ophthalmic lens of embodiment 57, wherein the second aspheric surface is a posterior surface of the lens body.
Embodiment 60. The ophthalmic lens of embodiment 57, wherein the first aspheric surface comprises a toric component.
Embodiment 61. A method of treating an ophthalmic disease or disorder in a subject, the method comprising implanting into an eye of the subject an ophthalmic lens comprising a lens body, the lens body comprising:
   a first aspheric surface; and
   a second aspheric surface comprising a base curvature and a phase-ring structure,
   wherein the phase-ring structure comprises an inner region, an intermediate region, and an outer region, and
      the inner region comprises a first phase-ring surface having a first curvature;
      the intermediate region comprises a second phase-ring surface having a second curvature;
      the outer region comprises a third phase-ring surface having a third curvature;
      an outer edge of the inner region is adjacent to an inner edge of the intermediate region; and
      an outer edge of the intermediate region is adjacent to an inner edge of the outer region,
   wherein refraction of light passing through the inner region, the intermediate region, and the outer region causes constructive interference that produces a near focus and a distance focus.
Embodiment 62. The method of embodiment 61, wherein the ophthalmic disease or disorder is selected from the group consisting of: cataract and presbyopia.
Embodiment 63. The method of embodiment 61, wherein the ophthalmic lens is an intraocular lens or a contact lens.
Embodiment 64. The method of embodiment 63, wherein the intraocular lens comprises a pair of haptics extended outwardly from the lens body.
Embodiment 65. The method of embodiment 63, wherein the intraocular lens is implanted into a capsular bag of the eye of the subject.
Embodiment 66. A method of manufacturing an ophthalmic lens, the method comprising:
   manufacturing a first aspheric surface;
   manufacturing a second aspheric surface comprising a base curvature; and
   generating a phase-ring structure on the second aspheric surface, wherein the phase-ring structure comprises an inner region, an intermediate region, and an outer region, and
      the inner region comprises a first phase-ring surface having a first curvature;
      the intermediate region comprises a second phase-ring surface having a second curvature;
      the outer region comprises a third phase-ring surface having a third curvature;
      an outer edge of the inner region is adjacent to an inner edge of the intermediate region; and
      an outer edge of the intermediate region is adjacent to an inner edge of the outer region,
   wherein refraction of light passing through the inner region, the intermediate region, and the outer region causes constructive interference that produces a near focus and a distance focus.
Embodiment 67. The method of embodiment 66, comprising performing an in-situ image quality analysis to determine whether a performance of the ophthalmic lens meets predetermined quality criteria.
Embodiment 68. A set of ophthalmic lenses, comprising:
   a first ophthalmic lens comprising a first lens body, the first lens body comprising:
      a first aspheric surface; and
      a second aspheric surface comprising a first base curvature and a first optical phase-ring structure, wherein the first optical phase-ring structure comprises a first inner region and a first outer region, and
         the first inner region comprises a first phase-ring surface; and
         the first outer region comprises a second phase-ring surface;
a second ophthalmic lens comprising a second lens body, the second lens body comprising:
   a third aspheric surface; and
   a fourth aspheric surface comprising a second base curvature and a second optical phase-ring structure, wherein the second optical phase-ring structure comprises a second inner region, an intermediate region, and a second outer region, and
      the second inner region comprises a third phase-ring surface;
      the intermediate region comprises a fourth phase-ring surface; and
      the second outer region comprises a fifth phase-ring surface;
wherein refraction of light passing through the first inner region and the outer region of the first ophthalmic lens causes first constructive interference and refraction of light passing through the second inner region, the intermediate region, and the second outer region causes second constructive interference, thereby collectively producing a near focus, distance focus, and an extended focus.

1. An ophthalmic lens comprising a lens body, the lens body comprising:
a first aspheric surface; and
a second aspheric surface comprising a base curvature and a phase-ring structure, wherein the phase-ring structure comprises an inner region and an outer region, and
the inner region comprises a first phase-ring surface having a first curvature;
the outer region comprises a second phase-ring surface having a second curvature; and
an outer edge of the inner region is adjacent to an inner edge of the outer region,
wherein refraction of light passing through the inner region and the outer region causes constructive interference that produces a distance focus (f₀) and an extended focus (f₁).

2. The ophthalmic lens of claim 1, wherein a radius of the inner region of the phase-ring structure that extends from an optical axis of the ophthalmic lens to the outer edge of the inner region is greater than 0 mm and less than or equal to 0.70 mm.

3. The ophthalmic lens of claim 1, wherein a radius of the outer region of the phase-ring structure that extends from an optical axis of the ophthalmic lens to an outer edge of the outer region is greater than 0 mm and less than or equal to 1.30 mm.

4. The ophthalmic lens of claim 1, wherein the first curvature of the first phase-ring surface comprises a first inner sloping portion that slopes at a first angle with respect to an optical axis of the ophthalmic lens and a first outer sloping portion that slopes at a second angle with respect to the optical axis.

5. The ophthalmic lens of claim 4, wherein the first inner sloping portion and the first outer sloping portion together slope monotonically with respect to the optical axis.

6. The ophthalmic lens of claim 1, wherein the second curvature of the second phase-ring surface comprises a second inner sloping portion that slopes at a third angle with respect to an optical axis of the ophthalmic lens, an intermediate sloping portion that slopes at a fourth angle with respect to the optical axis, and a second outer sloping portion that slopes at a fifth angle with respect to the optical axis.

7. The ophthalmic lens of claim 6, wherein the intermediate sloping portion and the second outer sloping portion together slope monotonically with respect to the optical axis, and the intermediate portion and the second inner sloping portion together do not slope monotonically with respect to the second outer sloping portion.

8. The ophthalmic lens of claim 1, wherein the first aspheric surface is an anterior surface of the lens body.

9. The ophthalmic lens of claim 1, wherein the second aspheric surface is a posterior surface of the lens body.

10. The ophthalmic lens of claim 1, wherein the first aspheric surface comprises a toric component.

11. The ophthalmic lens of claim 1, wherein a height profile (Zₐₛₚ) of the base curvature is represented by:
$${Z}_{asp} = \frac{{cr}^{2}}{1 + \sqrt{1 - ( 1 + k ) {c}^{2} {r}^{2}}} + {\sum }_{i = 2}^{n} {A}_{i} {r}^{2 i}$$
wherein r is a radial distance from the center of the lens in millimeters, c is a lens curvature, k is a conic constant, and Aᵢ is a higher order correction term.

12. The ophthalmic lens of claim 1, wherein a height profile of the lens body (Zₜₒₜₐₗ) is represented by:
$${Z}_{total} ( r ) = {Z}_{asp} ( r ) + {Z}_{phase} ( r ) .$$

13. The ophthalmic lens of claim 1, wherein the phase profile (Φ₍ₙ₎(r)) is represented by:
$${Φ}_{( n )} ( r ) = {A}_{( n )} \times f ( \frac{r - {r}_{n}}{{r}_{n + 1} - {r}_{n}} ) + {D}_{( n )} ,$$
wherein r is the radial distance from the center of the lens in millimeters, rₙ is a radius of an n^{th} zone, rₙ₊₁ is a radius of an (n+1)^{th} zone, f(r) is a phase function base profile, the phase function base profile being a linear, parabolic, sine or polynomial function, A₍ₙ₎ is a ratio of the phase function base profile, and D₍ₙ₎ is a phase shift of the phase function base profile.

14. The ophthalmic lens of claim 13, wherein the radius of the n^{th} zone (rₙ) is represented by:
$${r}_{n} = \sqrt{2 \times λ \times n \times f} ,$$
wherein λ is a design wavelength, and f is a reciprocal of add power.

15. The ophthalmic lens of claim 13, wherein a height ratio between the inner region and the outer region of the phase-ring structure is represented by:
$$0 \leq {Φ}_{( n )} ( {r}_{out} ) - {Φ}_{( n )} ( {r}_{in} ) \leq 4 π .$$

16. The ophthalmic lens of claim 1, wherein a height profile (Zₚₕₐₛₑ) of the phase-ring structure is relative to the base curvature of the ophthalmic lens:
$${Z}_{phase} ( r ) = {Φ}_{( n )} ( r ) \times \frac{λ}{{n}_{1} - {n}_{0}}$$
wherein λ is a design wavelength, Φ₍ₙ₎(r) is a phase profile, n₁ is a refractive index of a material of the ophthalmic lens, and no is a refractive index of a medium covering the ophthalmic lens.

17. The ophthalmic lens of claim 1, wherein a phase profile (Φ₍ₙ₎(r)) of the phase-ring structure is between:
$$0 \leq \vert {Φ}_{( n )} ( r ) \vert \leq 8 π .$$

18. The ophthalmic lens of claim 1, wherein the distance focus (f₀) and the extended focus (f₁) are defined by the following expressions:
$$- 15 D \leq \frac{1}{{f}_{0}} \leq 55 D ,$$
$$\frac{1}{6 f} \leq \frac{1}{{f}_{1}} - \frac{1}{{f}_{0}} \leq \frac{1}{2 f} ,$$
$$and$$
$$3 D \leq \frac{1}{f} \leq 16 D ,$$
wherein
$$\frac{1}{f}$$
is add power.

19. The ophthalmic lens of claim 1, wherein the ophthalmic lens is an intraocular lens or a contact lens.

20. The ophthalmic lens of claim 1, comprising a pair of haptics extended outwardly from the lens body.

21. A method of treating an ophthalmic disease or disorder in a subject, the method comprising implanting into an eye of the subject an ophthalmic lens comprising a lens body, the lens body comprising:
a first aspheric surface; and
a second aspheric surface comprising a base curvature and a phase-ring structure, wherein the phase-ring structure comprises an inner region and an outer region, and
the inner region comprises a first phase-ring surface having a first curvature;
the outer region comprises a second phase-ring surface having a second curvature; and
an outer edge of the inner region is adjacent to an inner edge of the outer region,
wherein refraction of light passing through the inner region and the outer region causes constructive interference that produces a distance focus and an extended focus.

22. The method of claim 21, wherein the ophthalmic disease or disorder is selected from the group consisting of: cataract and presbyopia.

23. The method of claim 21, wherein the ophthalmic lens is an intraocular lens or a contact lens.

24. The method of claim 23, wherein the intraocular lens comprises a pair of haptics extended outwardly from the lens body.

25. The method of claim 23, wherein the intraocular lens is implanted into a capsular bag of the eye of the subject.

26. A method of manufacturing an ophthalmic lens, the method comprising:
manufacturing a first aspheric surface;
manufacturing a second aspheric surface comprising a base curvature; and
generating a phase-ring structure on the second aspheric surface, wherein the phase-ring structure comprises an inner region and an outer region, and
the inner region comprises a first phase-ring surface having a first curvature;
the outer region comprises a second phase-ring surface having a second curvature; and
an outer edge of the inner region is adjacent to an inner edge of the outer region,
wherein refraction of light passing through the inner region and the outer region causes constructive interference that produces a distance focus and an extended focus.

27. The method of claim 26, comprising performing an in-situ image quality analysis to determine whether a performance of the ophthalmic lens meets predetermined quality criteria.

28. An ophthalmic lens comprising a lens body, the lens body comprising:
a first aspheric surface; and
a second aspheric surface comprising a base curvature and a phase-ring structure, wherein the phase-ring structure comprises an inner region, an intermediate region, and an outer region, and
the inner region comprises a first phase-ring surface having a first curvature;
the intermediate region comprises a second phase-ring surface having a second curvature;
the outer region comprises a third phase-ring surface having a third curvature;
an outer edge of the inner region is adjacent to an inner edge of the intermediate region; and
an outer edge of the intermediate region is adjacent to an inner edge of the outer region,
wherein refraction of light passing through the inner region, the intermediate region, and the outer region causes constructive interference that produces a near focus and a distance focus.

29. The ophthalmic lens of claim 28, wherein a radius of the inner region of the phase-ring structure that extends from an optical axis of the ophthalmic lens to the outer edge of the inner region is greater than 0 mm and less than or equal to 0.70 mm.

30. The ophthalmic lens of claim 28, wherein a radius of the intermediate region of the phase-ring structure that extends from an optical axis of the ophthalmic lens to the outer edge of the intermediate region is greater than 0 mm and less than or equal to 1.30 mm.

31. The ophthalmic lens of claim 28, wherein a radius of the outer region of the phase-ring structure that extends from an optical axis of the ophthalmic lens to an outer edge of the outer region is greater than 0 mm and less than or equal to 1.30 mm.

32. The ophthalmic lens of claim 28, wherein the first curvature of the first phase-ring surface comprises a first inner sloping portion that slopes at a first angle with respect to an optical axis of the ophthalmic lens and a first outer sloping portion that slopes at a second angle with respect to the optical axis.

33. The ophthalmic lens of claim 32, wherein the first inner sloping portion and the first outer sloping together portion slope monotonically with respect to the optical axis.

34. The ophthalmic lens of claim 28, wherein the second curvature of the second phase-ring surface comprises a second inner sloping portion that slopes at a third angle with respect to an optical axis of the ophthalmic lens and a second outer sloping portion that slopes at a fourth angle with respect to the optical axis.

35. The ophthalmic lens of claim 34, wherein the second inner sloping portion and the second outer sloping portion together do not slope monotonically with respect to the optical axis.

36. The ophthalmic lens of claim 28, wherein the third curvature of the third phase-ring surface comprises a third inner sloping portion that slopes at a fifth angle with respect to an optical axis of the ophthalmic lens and a third outer sloping portion that slopes at a sixth angle with respect to the optical axis.

37. The ophthalmic lens of claim 36, wherein the third inner sloping portion and the third outer sloping portion together do not slope monotonically with respect to the optical axis.

38. The ophthalmic lens of claim 28, wherein the first aspheric surface is an anterior surface of the lens body.

39. The ophthalmic lens of claim 28, wherein the second aspheric surface is a posterior surface of the lens body.

40. The ophthalmic lens of claim 28, wherein the first aspheric surface comprises a toric component.

41. A method of treating an ophthalmic disease or disorder in a subject, the method comprising implanting into an eye of the subject an ophthalmic lens comprising a lens body, the lens body comprising:
a first aspheric surface; and
a second aspheric surface comprising a base curvature and a phase-ring structure, wherein the phase-ring structure comprises an inner region, an intermediate region, and an outer region, and
the inner region comprises a first phase-ring surface having a first curvature;
the intermediate region comprises a second phase-ring surface having a second curvature;
the outer region comprises a third phase-ring surface having a third curvature;
an outer edge of the inner region is adjacent to an inner edge of the intermediate region; and
an outer edge of the intermediate region is adjacent to an inner edge of the outer region,
wherein refraction of light passing through the inner region, the intermediate region, and the outer region causes constructive interference that produces a near focus and a distance focus.

42. The method of claim 41, wherein the ophthalmic disease or disorder is selected from the group consisting of: cataract and presbyopia.

43. The method of claim 41, wherein the ophthalmic lens is an intraocular lens or a contact lens.

44. The method of claim 43, wherein the intraocular lens comprises a pair of haptics extended outwardly from the lens body.

45. The method of claim 43, wherein the intraocular lens is implanted into a capsular bag of the eye of the subject.

46. A method of manufacturing an ophthalmic lens, the method comprising:
manufacturing a first aspheric surface;
manufacturing a second aspheric surface comprising a base curvature; and
generating a phase-ring structure on the second aspheric surface, wherein the phase-ring structure comprises an inner region, an intermediate region, and an outer region, and
the inner region comprises a first phase-ring surface having a first curvature;
the intermediate region comprises a second phase-ring surface having a second curvature;
the outer region comprises a third phase-ring surface having a third curvature;
an outer edge of the inner region is adjacent to an inner edge of the intermediate region; and
an outer edge of the intermediate region is adjacent to an inner edge of the outer region,
wherein refraction of light passing through the inner region, the intermediate region, and the outer region causes constructive interference that produces a near focus and a distance focus.

47. The method of claim 46, comprising performing an in-situ image quality analysis to determine whether a performance of the ophthalmic lens meets predetermined quality criteria.

48. A set of ophthalmic lenses, comprising:
a first ophthalmic lens comprising a first lens body, the first lens body comprising:
a first aspheric surface; and
a second aspheric surface comprising a first base curvature and a first optical phase-ring structure, wherein the first optical phase-ring structure comprises a first inner region and a first outer region, and
the first inner region comprises a first phase-ring surface; and
the first outer region comprises a second phase-ring surface;
a second ophthalmic lens comprising a second lens body, the second lens body comprising:
a third aspheric surface; and
a fourth aspheric surface comprising a second base curvature and a second optical phase-ring structure, wherein the second optical phase-ring structure comprises a second inner region, an intermediate region, and a second outer region, and
the second inner region comprises a third phase-ring surface;
the intermediate region comprises a fourth phase-ring surface; and
the second outer region comprises a fifth phase-ring surface;
wherein refraction of light passing through the first inner region and the outer region of the first ophthalmic lens causes first constructive interference and refraction of light passing through the second inner region, the intermediate region, and the second outer region causes second constructive interference, thereby collectively producing a near focus, distance focus, and an extended focus.
